(19) United States
(12) Patent Application Publication
(10) Pub. No.: US 2026/0263156 A1
(43) Pub. Date: Sep. 10, 2026

(54) METHODS FOR PRE-OPERATIVE LUNG ABLATION PREDICTION USING DEEP LEARNING

(71) Applicants: Memorial Sloan-Kettering Cancer Center, New York, NY (US); Memorial Hospital for Cancer and Allied Diseases, New York, NY (US); Sloan-Kettering Institute for Cancer Research, New York, NY (US)

(72) Inventors: Etay Ziv, New York, NY (US); Krishna Nand Keshavamurthy, New York, NY (US)

(21) Appl. No.: 19/491,315

(22) PCT Filed: Jun. 7, 2024
(86) PCT No.: PCT/US2024/033051

§ 371 Date: Dec. 8, 2025

Related U.S. Application Data
(60) Provisional application No. 63/507,356, filed on Jun. 9, 2023.
(60) Provisional application No. 63/626,367, filed on Jan. 29, 2024.

(51) Int. Cl. A61B 34/10; A61B 18/00; G06T 7/00; G06T 7/33; G06V 10/70; G06V 20/70

(52) U.S. Cl. CPC A61B 34/10; A61B 18/00; G06T 7/0012; G06T 7/337; G06V 10/70; G06V 20/70; A61B 2018/00577; A61B 2034/104; A61B 2034/107; G06T 2207/20081; G06T 2207/30061; G06T 2207/30096; G06V 2201/031

(57) ABSTRACT

The present disclosure relates generally to methods and systems for predicting pre-operative lung ablation in lung cancer patients in need thereof and the application of machine learning to perform microwave lung ablation with accurate margins to prevent local tumor recurrence.

### CROSS-REFERENCE TO RELATED APPLICATIONS

This application claims the benefit of and priority to U.S. Provisional Patent Application No. 63/507,356, filed Jun. 9, 2023, and U.S. Provisional Patent Application No. 63/626,367, filed Jan. 29, 2024, the contents of which are incorporated herein by reference in their entireties.

### GOVERNMENT SUPPORT

This invention was made with government support under CA008748 awarded by the National Institutes of Health. The government has certain rights in the invention.

### TECHNICAL FIELD

The present disclosure relates generally to methods and systems for predicting pre-operative lung ablation in lung cancer patients in need thereof and the application of machine learning to perform microwave lung ablation with accurate margins to prevent local tumor recurrence.

### BACKGROUND

The following description of the background of the present technology is provided simply as an aid in understanding the present technology and is not admitted to describe or constitute prior art to the present technology.

Microwave lung ablation (MWA) is an effective local therapy to eradicate tumors in the lung.¹ The procedure is minimally invasive and often performed in an outpatient setting and thus offers a low morbidity and inexpensive alternative to surgical resection for patients with comorbidities or with multiple tumors.² Moreover, there are no detectable long-term effects on pulmonary function, making this an attractive alternative to stereotactic body radiation therapy.³ However, widespread adaptation of MWA for cancer therapy has been hindered by higher local recurrence rates.⁴ Local recurrence is associated with incomplete ablations with insufficient margins.⁵

Unfortunately, achieving adequate margins with MWA is challenging. In a retrospective study, only 11% of lung ablations had adequate margins (>5 mm).⁵ The ablation device vendors provide a chart of the expected ablation zone dimensions as a function of power and duration (henceforth referred to as the vendor model), which is used for pre-procedure planning. The vendor model is based on ex vivo experimental observations on bovine or swine organs and are ellipsoids of varying dimensions. Review of clinically observed ablation zones have demonstrated deviation from the vendor models (45% of the observed treatment volumes deviated by more than 50% from their vendor predicted volumes).^{6,7} As a result, the treatment plan based on the vendor model may significantly differ from the actual treatment, potentially leading to incomplete thermal destruction of tumors and failure to establish an adequate margin. These problems are further exacerbated in the lung due to multiple large vessels and airways acting as heat sinks, causing distortions in the ablation zone, and large and complex deformations of the lung due to patient motion and breathing. These challenges translate directly to recurrence rates as high as 30% compared with 5% for colorectal liver thermal ablation.^{4,17}

There have been several attempts to simulate ablation zones using biophysical models, specifically by solving partial differential equations governing the physical processes during MWA including electromagnetic wave propagation and absorption in tissue, heat transfer in tissue, and thermal tissue damage.^{8,9} However, these models are limited a) in their ability to account for patient-specific tissue properties and their time dependence during an ablation, b) in their ability to account for effects of varying anatomical geometry such as organ/lobar boundaries, c) by lack of clinical validation rather than simulation models and d) by computational complexity that precludes implementation in real-time clinical settings.

Accordingly, there is an urgent need for accurate and sensitive methods for predicting pre-operative lung ablation in cancer patients.

### SUMMARY

Aspects of the present disclosure are directed to systems, methods, and non-transitory computer readable media for training models to determine ablation regions from biomedical images of subjects. A computing system may identify, for a subject under treatment for lung cancer: (i) a first biomedical image having a first region of interest (ROI) corresponding to a tumor in a lung region prior to an ablation procedure, (ii) a second biomedical image having a second ROI corresponding to the administration of the ablation procedure in the lung region, (iii) a parameter defining an administration of the ablation procedure to the tumor in the lung region, and (iv) an annotation defining a first ablation region identifying a segment within the second biomedical image corresponding to the administration of the ablation. The computing system may register the first biomedical image with the second biomedical image using the first ROI and the second ROI to generate a third biomedical image. The computing system may apply a machine learning (ML) to the third biomedical image and the parameter to determine a second ablation region to identify a segment within the second biomedical image corresponding to the administration of the ablation. The computing system may determine a loss metric based on a comparison between the first ablation region and the second ablation region. The computing system may update at least one weight of the ML model in accordance with the loss metric.

In some embodiments, the computing system may convert, prior to applying the ML model, the third biomedical image and the parameter to a coordinate system defined relative to an applicator within the lung region to administer the ablation procedure. In some embodiments, the computing system may generate, within the third biomedical image, an application region identifying an application of the ablation procedure in the lung region based on the parameter. In some embodiments, the computing system may apply the ML model to the application region to determine the second ablation region.

In some embodiments, the computing system may constrain the registration of the first biomedical image with the second biomedical image as a function of a first anatomical structure corresponding to the first ROI, a second anatomical structure corresponding to the second ROI, and the administration of the ablation procedure. In some embodiments, the annotation may identify the first ablation region does not correspond to any anatomical boundary associated with the lung region of the subject.

In some embodiments, the computing system may register the first biomedical image with the second biomedical image in accordance with a sequence of image registration operations. The sequence of image registration operations may include one or more of: (i) an affine registration between the first biomedical image and the second biomedical image, (ii) a first deformable image registration between the first biomedical image and the second biomedical image, (iii) a second deformable image registration between a first portion of the first biomedical image corresponding to the lung region and a second portion of the second biomedical image corresponding to the lung region, (iv) a third deformable image registration between the first ROI of the first biomedical image and the second ROI of the second biomedical image, or (v) a landmark-based registration between a first portion of the first biomedical image corresponding to a first anatomical structure in the lung region and a second portion of the second biomedical image corresponding to a second anatomical structure in the lung region.

In some embodiments, the parameter may include at least one of: (i) a position of the applicator within the first biomedical image, (ii) a power to apply to the tumor during the ablation procedure, (iii) a duration of the administration of the ablation procedure, and (iv) a trajectory of the applicator through the lung region. In some embodiments, the ablation procedure may include at least one of: (i) a radiofrequency ablation (RFA), (ii) a microwave ablation (MWA), or (iii) a cryoablation.

Aspects of the present disclosure are directed to systems, methods, and non-transitory computer readable media for determining ablation regions from biomedical images of subjects. A computing system may identify, for a first subject under treatment for lung cancer: (i) a first biomedical image having a first region of interest (ROI) corresponding to a tumor in a lung region prior to an ablation procedure and (ii) a parameter defining an administration of the ablation procedure to the tumor within the lung region. The computing system may apply a machine learning (ML) model to the first biomedical image and the parameter to determine an ablation region to identify a segment within the first biomedical image corresponding to an administration of the ablation procedure. The computing system may store, using one or more data structures, an association between the first subject and the ablation region.

In some embodiments, the computing system may convert, prior to applying the ML model, the first biomedical image and the parameter to a coordinate system defined relative to an applicator within the lung region to administer the ablation procedure. In some embodiments, the computing system may generate, within the first biomedical image, an application region identifying an application of the ablation procedure in the lung region based on the parameter. In some embodiments, the computing system may apply the ML model to the application region to determine the ablation region.

In some embodiments, the computing system may provide information for presentation based on the association between the first subject and the ablation region. In some embodiments, the computing system may select, via an interface, the parameter from a plurality of parameters to determine the ablation region in the subject in need of the ablation procedure, prior to the administration of the ablation procedure.

In some embodiments, the parameter may include at least one of: (i) a position of an applicator within the first biomedical image, (ii) a power to apply to the tumor during the ablation procedure, (iii) a duration of the administration of the ablation procedure, and (iv) a trajectory of the applicator through the lung region. In some embodiments, the ablation procedure may include at least one of: (i) a radiofrequency ablation (RFA), (ii) a microwave ablation (MWA), or (iii) a cryoablation. In some embodiments, the ablation procedure may be performed in accordance with the determined ablation region. In some embodiments, the ablation procedure may be planned in accordance with the determined ablation region.

### BRIEF DESCRIPTION OF THE DRAWINGS

FIG. 1 depicts a block diagram of a system for determining ablation regions from biomedical images of subjects, in accordance with an illustrative embodiment.

FIGS. 2A and 2B depict a block diagrams of a process for training an ablation prediction model in a system for determining ablation regions in accordance with an illustrative embodiment.

FIG. 3 depicts a block diagram of an architecture for an ablation prediction model in a system for determining ablation regions in accordance with an illustrative embodiment.

FIG. 4A depicts a block diagram of an architecture of a transform block in an ablation prediction model in a system for determining ablation regions in accordance with an illustrative embodiment.

FIG. 4B depicts a block diagram of an architecture of a set of transform layers in a transform block in an ablation prediction model in the system for determining ablation regions in accordance with an illustrative embodiment.

FIGS. 5A and 5B depict block diagrams of a process for applying an ablation prediction model to acquired biomedical images in the system for determining ablation regions in accordance with an illustrative embodiment.

FIG. 6 depicts a flow diagram of a method of determining ablation regions from biomedical images of subjects in accordance with an illustrative embodiment.

FIG. 7 depicts a flow diagram of a method of training models to determine ablation regions from biomedical images of subjects in accordance with an illustrative embodiment.

FIG. 8 depicts block diagram of a server system and a client computer system in accordance with an illustrative embodiment.

FIG. 9: Flow diagram of a work flow for predicting ablation zones in lung computed tomography (CT) scans.

FIG. 10: Flow diagram of number of participants and exclusions.

FIG. 11A shows ablation zone volume variability for the same power (65 W) and duration (5 min) of ablation. FIGS. 11B-11C show various pre-processing steps: segmentation of tumor in pre-procedure and ablation zone in follow-up post scans, and deformable image registration between pre- and post-is shown in FIG. 11B; illustration of applicator shadow on follow-up post scan is shown in (FIG. 11C) (top); applicator-centric coordinate system is shown in (FIG. 11C) (bottom). FIG. 11D shows the neural network model of the present technology.

FIGS. 12A-12C show Bland-Altman plots comparing the volumes and shapes of prediction model disclosed herein and vendor model with ground truth. Comparison of volumes is shown in (FIG. 12A), sphericity in (FIG. 12B), and surface-to-volume ratio in (FIG. 12C). The bias (mean difference) is shown by the solid line and the limits of agreement (1.96 SD of the difference on either side of the bias) are shown by dotted lines in each case.

FIGS. 13A-13C show boxplots of (FIG. 13A) Dice scores, (FIG. 13B) precision, and (FIG. 13C) recall quantifying overlap between predicted and ground truth. Each box extends from the lower to the upper quartile values, with median shown in orange. Notches around the median represent the 95% confidence interval. Dice score, precision, and recall were 0.62, 0.65, and 0.70, respectively, for the model described herein compared with 0.56, 0.43, and 0.89, respectively, for the vendor model.

FIGS. 14A-14D show examples demonstrating the ability of the method disclosed herein to predict ablation zones in various challenging scenarios. Two examples (on left and right) are shown for each scenario, with the prediction model (inner dotted line), vendor model (outer solid line), and true ablation (inner dashed line) overlaid on both the pre- and the follow-up scans in each example. Note that, unlike the vendor model, the method of the present technology accounts for the neighboring anatomical effects and is closer to the ground truth.

FIGS. 15A-15D demonstrate the ability of the methods of the present technology to predict ablation zones in additional challenging scenarios. Two examples (on left and right) are shown for each scenario, with the prediction model (inner dotted line), vendor model (outer solid line), and true ablation (inner dashed line) overlaid on both the pre- and the follow-up scans in each example. Some challenging cases that implemented the methods disclosed herein are also shown in FIG. 15C (right) and FIG. 15D.

FIG. 16 shows the effect of misregistration on Dice score. A reference ellipsoid is placed at the origin with major axis lying along the x axis. New ellipsoids are created (e.g., new ellipsoids A and B) that are translated and rotated (straight arrows along the x-axis and z-axis and rotating arrows about the x-, y-, and z-axes) and their Dice scores with the reference computed.

FIG. 17: Older and newer registration methods compared using target registration error (TRE). Lower TRE indicates higher registration accuracy. The newer registration resulted in median TRE of 0.297 mm, which is ~8 times improvement over the older method with TRE of 2.26 mm, p<0.01.

FIG. 18: Top panel shows the tumor in pre scan (rectangle) registered to the ablation zone in the post procedure scan (dashed oval). The registration was performed using the newer registration method. We observe that the tumor extends beyond the ablated area, indicating residual disease after treatment and poor treatment margins. Bottom panel shows that poor margins (>5 mm) are predictive of local tumor recurrence (p<0.05) post procedure. The margins were computed in 3D using the method based on the minimum function (Salkin R, Keshavamurthy K N, Dev A, et al. Abstract No. 127, Journal of Vascular and Interventional Radiology. 2022; 33(6):S59-S60), which measures the volumetric minimum distance between the tumor and ablation zone surfaces after registering the pre and post procedure scans in the data set.

FIG. 19: The ablation prediction algorithm described herein has much higher dice score compared to the vendor model when close to large blood vessels. This indicates the ability of the algorithm described herein and the inability of the vendor model to account for local anatomical effects, in this case heat sink effects due to vessels.

FIG. 20: GradCam saliency maps (heatmap) of the prediction of the present technology confirms that the appropriate local anatomical features most influenced the output (local tumor neighborhood, vessels and chest wall).

### DETAILED DESCRIPTION

It is to be appreciated that certain aspects, modes, embodiments, variations and features of the present methods are described below in various levels of detail in order to provide a substantial understanding of the present technology.

In practicing the present methods, many conventional techniques in molecular biology, protein biochemistry, cell biology, immunology, microbiology and recombinant DNA are used. See, e.g., Sambrook and Russell eds. (2001) Molecular Cloning: A Laboratory Manual, 3^{rd} edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5^{th} edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology. Methods to detect and measure levels of polypeptide gene expression products (i.e., gene translation level) are well-known in the art and include the use of polypeptide detection methods such as antibody detection and quantification techniques. (See also, Strachan & Read, Human Molecular Genetics, Second Edition. (John Wiley and Sons, Inc., NY, 1999)).

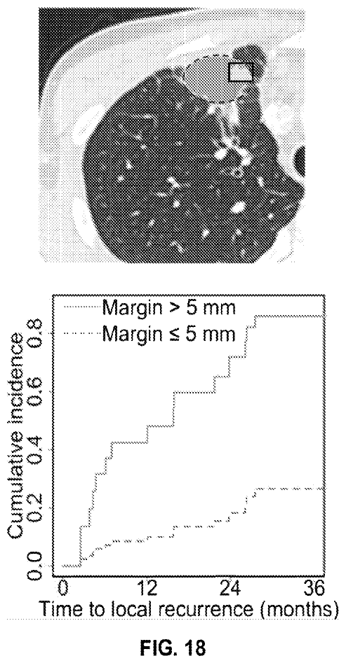

Disclosed herein is a deep learning model to predict lung ablation zones as they appear on the follow-up scan based on pre-procedure imaging data and ablation parameters. The model of the present technology demonstrate higher Dice scores compared with the vendor model with an 11% improvement. Moreover, the models of the present technology show no bias from ground truth volumes (p=0.169) unlike vendor's estimate (p<0.001) and had smaller limits of agreement (p<0.001) and bias (p<0.001) in both volume and shape characteristics. The model disclosed herein is also able to accurately predict the ablation zone in the context of challenging scenarios including local and global heat sink effects from vessels and airways, lung anatomic boundaries even when such boundaries were not readily visible on the input images, and variations in the shape of the ablation zone including uniform narrowing, asymmetric narrowing, and tilting. As shown in the Examples herein, the ability to account for patient-specific in vivo anatomical factors, such as vessels, chest wall, heart, lung boundaries, and fissures was demonstrated. These results demonstrate that the prediction models described herein are useful for facilitates microwave lung ablation with accurate margins to prevent local tumor recurrence. See FIG. 18 demonstrating that poor margins (>5 mm) are predictive of local tumor recurrence (p<0.05) post procedure.

### Definitions

Unless defined otherwise, all technical and scientific terms used herein generally have the same meaning as commonly understood by one of ordinary skill in the art to which this technology belongs. As used in this specification and the appended claims, the singular forms “a”, “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

As used herein, the term “about” in reference to a number is generally taken to include numbers that fall within a range of 1%, 5%, or 10% in either direction (greater than or less than) of the number unless otherwise stated or otherwise evident from the context (except where such number would be less than 0% or exceed 100% of a possible value).

As used herein, the “administration” of an agent or drug to a subject includes any route of introducing or delivering to a subject a compound to perform its intended function. Administration can be carried out by any suitable route, including but not limited to, orally, intranasally, parenterally (intravenously, intramuscularly, intraperitoneally, or subcutaneously), rectally, intrathecally, intratumorally or topically. Administration includes self-administration and the administration by another.

As used herein, the term “biological sample” means sample material derived from living cells. Biological samples may include tissues, cells, protein or membrane extracts of cells, and biological fluids (e.g., ascites fluid or cerebrospinal fluid (CSF)) isolated from a subject, as well as tissues, cells and fluids present within a subject. Biological samples of the present technology include, but are not limited to, samples taken from breast tissue, renal tissue, the uterine cervix, the endometrium, the head or neck, the gallbladder, parotid tissue, the prostate, the brain, the pituitary gland, kidney tissue, muscle, the esophagus, the stomach, the small intestine, the colon, the liver, the spleen, the pancreas, thyroid tissue, heart tissue, lung tissue, the bladder, adipose tissue, lymph node tissue, the uterus, ovarian tissue, adrenal tissue, testis tissue, the tonsils, thymus, blood, hair, buccal, skin, serum, plasma, CSF, semen, prostate fluid, seminal fluid, urine, feces, sweat, saliva, sputum, mucus, bone marrow, lymph, and tears. Biological samples can also be obtained from biopsies of internal organs or from cancers. Biological samples can be obtained from subjects for diagnosis or research or can be obtained from non-diseased individuals, as controls or for basic research. Samples may be obtained by standard methods including, e.g., venous puncture and surgical biopsy. In certain embodiments, the biological sample is a tissue sample obtained by needle biopsy.

As used herein, a “control” is an alternative sample used in an experiment for comparison purpose. A control can be “positive” or “negative.” For example, where the purpose of the experiment is to determine a correlation of the efficacy of a therapeutic agent for the treatment for a particular type of disease, a positive control (a compound or composition known to exhibit the desired therapeutic effect) and a negative control (a subject or a sample that does not receive the therapy or receives a placebo) are typically employed.

As used herein, the term “effective amount” refers to a quantity sufficient to achieve a desired therapeutic and/or prophylactic effect, e.g., an amount which results in the prevention of, or a decrease in a disease or condition described herein or one or more signs or symptoms associated with a disease or condition described herein. In the context of therapeutic or prophylactic applications, the amount of a composition administered to the subject will vary depending on the composition, the degree, type, and severity of the disease and on the characteristics of the individual, such as general health, age, sex, body weight and tolerance to drugs. The skilled artisan will be able to determine appropriate dosages depending on these and other factors. The compositions can also be administered in combination with one or more additional therapeutic compounds. In the methods described herein, the therapeutic compositions may be administered to a subject having one or more signs or symptoms of a disease or condition described herein. As used herein, a “therapeutically effective amount” of a composition refers to composition levels in which the physiological effects of a disease or condition are ameliorated or eliminated. A therapeutically effective amount can be given in one or more administrations.

As used herein, the terms “individual”, “patient”, or “subject” can be an individual organism, a vertebrate, a mammal, or a human. In some embodiments, the individual, patient or subject is a human.

As used herein, “prevention” or “preventing” of a disorder or condition refers to a compound that, in a statistical sample, reduces the occurrence of the disorder or condition in the treated sample relative to an untreated control sample, or delays the onset of one or more symptoms of the disorder or condition relative to the untreated control sample.

As used herein, the term “therapeutic agent” is intended to mean a compound that, when present in an effective amount, produces a desired therapeutic effect on a subject in need thereof.

“Treating” or “treatment” as used herein covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder. In some embodiments, treatment means that the symptoms associated with the disease are, e.g., alleviated, reduced, cured, or placed in a state of remission.

It is also to be appreciated that the various modes of treatment or prevention of disorders as described herein are intended to mean “substantial,” which includes total but also less than total treatment, and wherein some biologically or medically relevant result is achieved. The treatment may be a continuous prolonged treatment for a chronic disease or a single, or few time administrations for the treatment of an acute condition.

Systems and Methods for Determining Ablation Regions from Biomedical Images

The present disclosure generally relates to systems and methods for determining ablation regions in biomedical images of lung regions from subjects. A computing system may identify a pre-procedure image and a post-procedure image of a lung region in a subject. Between the acquisition of the two images, the subject may have undergone an ablation procedure, with an energy (e.g., in accordance with microwave ablation) applied to a tumor within the lung region. With the identification, the computing system may register the two images based on anatomical structures and other visual characteristics in the images. Using the correspondences determined from the registration, the computing system may modify the pre-procedure image to account for the anatomical changes between the images. The computing system may also identify parameters defining the administration of the ablation procedure, such as power, duration, and position of the applicator. The computing service may apply a machine learning (ML) model to the modified pre-procedure image to determine a predicted ablation region in the lung region as depicted in the post-procedure image. The ML model may provide for more accurate and quicker prediction of the ablation region between the pre-procedure and post-procedure image.

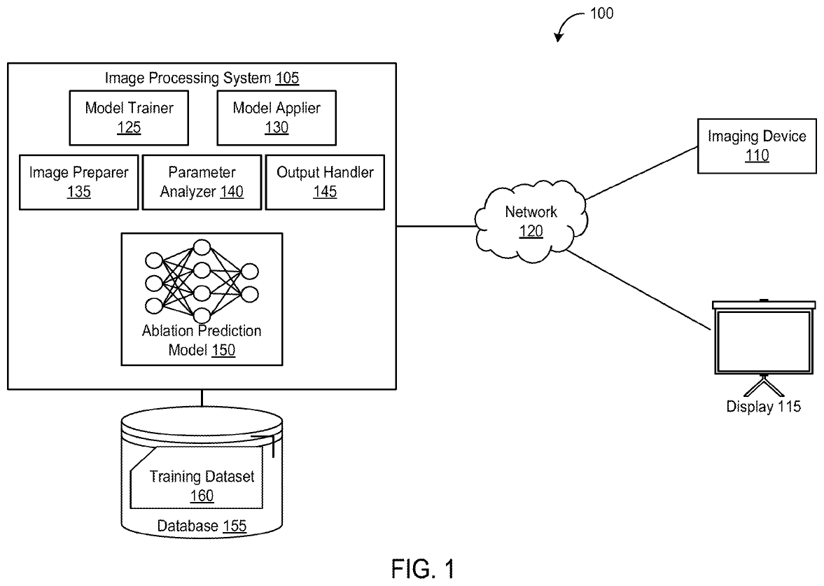

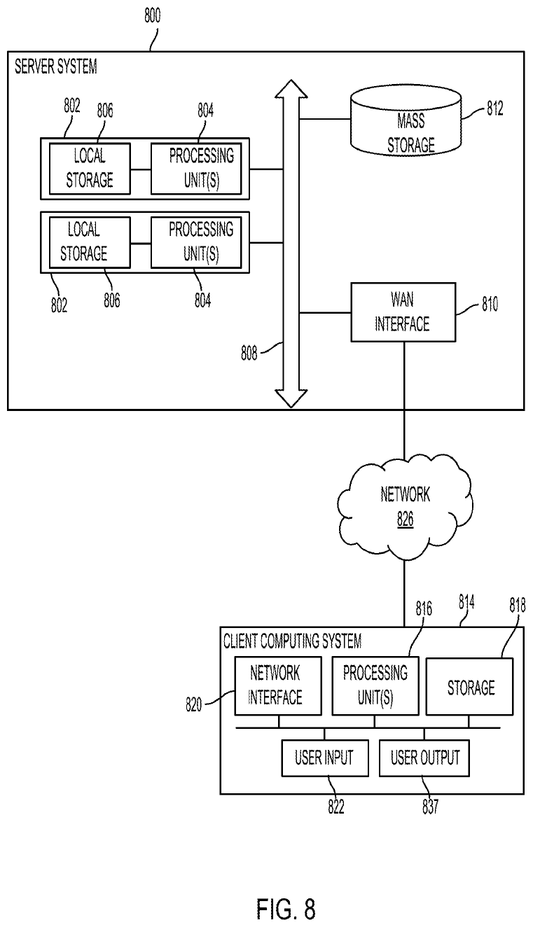

Referring now to FIG. 1, depicted is a block diagram of a system 100 for determining ablation regions from biomedical images of subjects. In overview, the system 100 may include at least one image processing system 105, at least one imaging device 110, and at least one display 115, communicatively coupled via at least one network 120. The image processing system 105 may include at least one model trainer 125, at least one model applier 130, at least one image preparer 135, at least one parameter analyzer 140, at least one output handler 145, at least one ablation prediction model 150, and at least one database 155, among others. The database 155 may include at least one training dataset 160. Each of the components in the system 100 as detailed herein may be implemented using hardware (e.g., one or more processors coupled with memory) or a combination of hardware and software as detailed herein in conjunction with FIG. 8. Each of the components in the system 100 may implement or execute the functionalities detailed herein, such as those described in Examples 1-3.

In further detail, the image processing system 105 may (sometimes herein generally referred to as a computing system or a server) be any computing device comprising one or more processors coupled with memory and software and capable of performing the various processes and tasks described herein. The image processing system 105 may be in communication with the network 120. The image processing system 105 may be situated, located, or otherwise associated with at least one server group. The server group may correspond to a data center, a branch office, or a site at which one or more servers corresponding to the image processing system 105 is situated.

Within the image processing system 105, the model trainer 125 may initialize, train, and establish the ablation prediction model 150 using the training dataset 160. The model applier 130 may apply biomedical images of lung regions of subjects before and after ablation procedures and parameter sets defining the administration of the ablation procedures to the ablation prediction model 150. The image preparer 135 may perform processing (e.g., image registration) on the biomedical images of lung regions of subjects before and after ablation procedures for input into the ablation prediction model 150. The parameter analyzer 140 may perform processing on the parameter sets defining the administration of the ablation procedures to the ablation prediction model 150. The output handler 145 may generate information for presentation using the output of the ablation prediction model 150.

The image processing system 105 itself and the components therein, such as the model trainer 125, the model applier 130, the image preparer 135, the parameter analyzer 140, and the output handler 145, and the ablation prediction model 150 may have a training mode and a runtime mode (sometimes herein referred to as an evaluation or inference mode). Under the training mode, the image processing system 105 may invoke the model trainer 125 to train the ablation prediction model 150 using the training dataset 160 (e.g., in accordance with supervised learning techniques). Under the runtime, the image processing system 105 may invoke the model applier 130 to apply the ablation prediction model 150 to acquired images from the imaging device 110.

The imaging device 110 (sometimes herein generally referred to as an imaging device or an image acquirer) may be any device for acquiring biomedical images of lung regions of subjects. The subjects may have been diagnosed with lung cancer, and may be undergoing treatment for lung cancer or may be under preparation to receive treatment. The treatment procedure to address the lung cancer may be an ablation procedure to eliminate tumor within the lung region. The subjects may be under guidance of clinician or hospital staff while scanned by the imaging device 110. The imaging device 110 may perform the scan in accordance with any number of imaging modalities, such as X-ray scan, a computed tomography (CT) scan, a computed tomography laser mammography (CTLM), a magnetic resonance imaging (MRI) scan, a nuclear magnetic resonance (NMR) scan, an ultrasound imaging scan, a positron emission tomography (PET) scan, or a photoacoustic spectroscopy scan, among others.

The display 115 may be communicatively coupled with the image processing system 105 or any other computing device comprising one or more processors coupled with memory and software and capable of performing the various processes and tasks described herein. The display 115 may display, render, or otherwise present any information provided by the image processing system 105 or the images of subjects acquired via the imagining device 110. The information may be used by a clinician examining a subject in diagnosing and deciding how to carry out the ablation procedure treatment to administer to the subject.

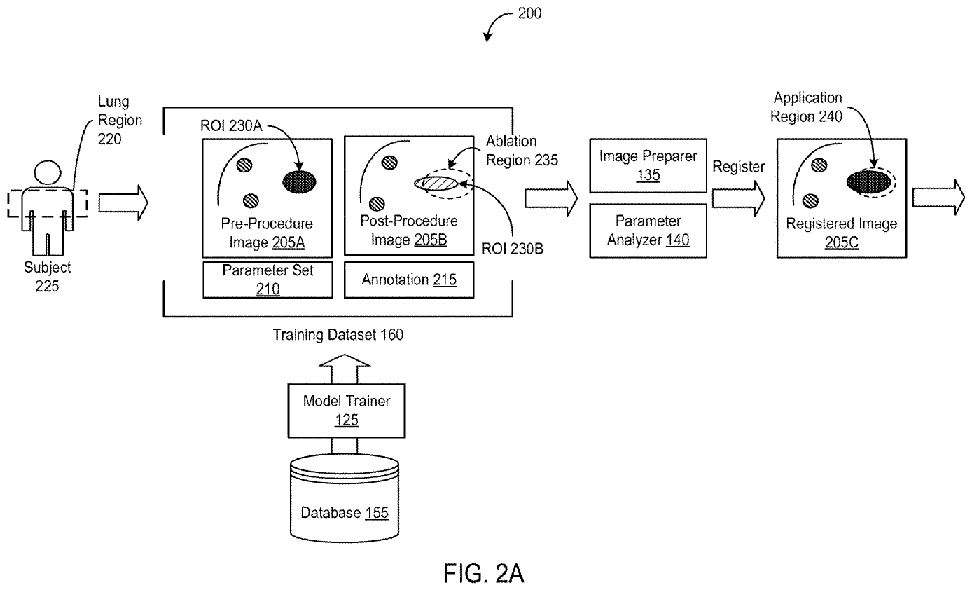

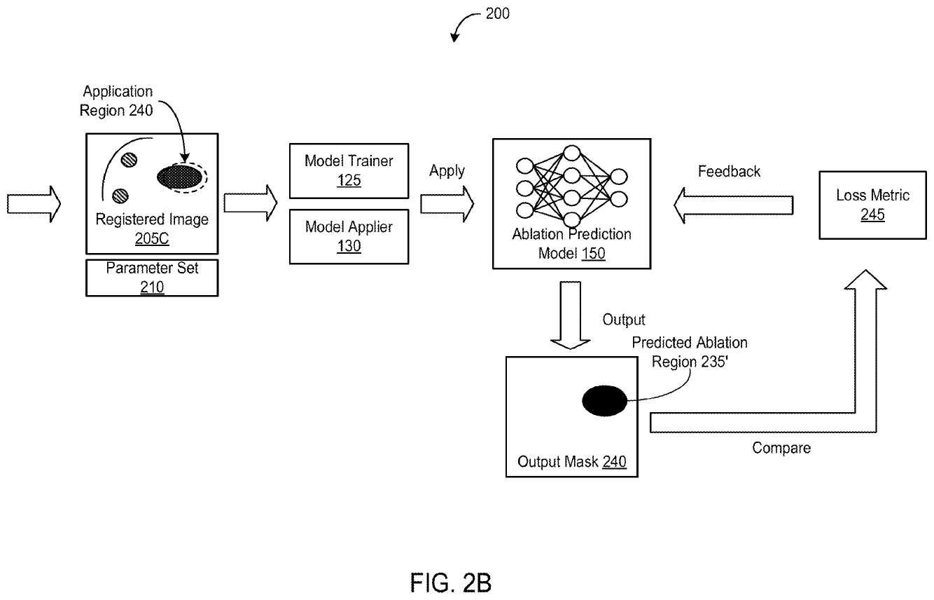

Referring now to FIGS. 2A and 2B depict a block diagram of a process 200 for training an ablation prediction model in the system 100 for determining ablation regions. The process 200 may include or correspond to operations performed in the system 100 for training the ablation prediction model 150. Starting from FIG. 2A, under the process 200, the model trainer 125 executing on the image processing system 105 may retrieve, receive, or otherwise identify the training dataset 160 to be used to train the ablation prediction model 150. In some embodiments, the model trainer 125 may access the database 155 to fetch, retrieve, or identify the training dataset 160. In some embodiments, the model trainer 125 may identify or select a subset of examples from the training dataset 160 to use to train the ablation prediction model 150. For instance, the model trainer 125 may select 60%-80% of the examples from the training dataset 160 to train the ablation prediction model 150, while setting aside the remainder of examples in the training dataset 160 for validation.

The training dataset 160 may identify or include a set of examples. Each example may identify or include at least one pre-procedure image 205A (sometimes referred herein as a first biomedical image, an image, or a tomogram), at least one post-procedure image 205B (sometimes referred herein as a second biomedical image, an image, or a tomogram), at least one parameter set 210 (sometimes referred herein as ablation parameters), and at least one annotation 215, among others. Each example may correspond to a lung region 220 of a respective subject 225 under treatment for lung cancer via administration of an ablation procedure. The lung cancer may be, for example, one of the following: non-small cell lung cancer (NSCLC) (e.g., adenocarcinoma, squamous cell carcinoma, or large cell carcinoma) or small cell lung cancer (SCLS), among others.

In each example, the pre-procedure image 205A and the post-procedure image 205B may each derived, acquired, or otherwise be of at least a portion the lung region 220 of the subject 225 (e.g., a human patient or an animal). The lung region 220 may generally correspond to a portion or a volume of the subject 225 in which at least one lung is located. For example, the lung region 220 may correspond a chest cavity of the subject 225 in which one or both pair of lungs are located. Both the pre-procedure image 205A and the post-procedure image 205B may capture at least one tumor associated with the incidence of lung cancer within the lung region 220. The pre-procedure image 205A may be acquired prior to the administration of the ablation procedure (e.g., a day to a month before the procedure). The post-procedure image 205B may be acquired subsequent to the administration of the ablation procedure (e.g., a week to one or two months after the procedure).

The pre-procedure image 205A and the post-procedure image 205B may each be acquired using any number of imaging modalities in accordance with lung cancer screening techniques. For example, each of the pre-procedure image 205A and the post-procedure image 205B may include an X-ray scan, a computed tomography (CT) scan, a computed tomography laser mammography (CTLM), a magnetic resonance imaging (MRI) scan, a nuclear magnetic resonance (NMR) scan, an ultrasound imaging scan, a positron emission tomography (PET) scan, or a photoacoustic spectroscopy scan, among others, of the lung region 220 of the subject 225. The pre-procedure image 205A and the post-procedure image 205B may each include a set of two-dimensional cross-sections (e.g., a front, a sagittal, a transverse, or an oblique plane) acquired from the three-dimensional volume. The pre-procedure image 205A and the post-procedure image 205B each may be defined in terms of pixels, in two-dimensions or three-dimensions. Although primarily discussed in terms of CT scans, other imaging modalities besides those listed above may be supported by the image processing system 105 for the pre-procedure image 205A and the post-procedure image 205B. The pre-procedure image 205A and the post-procedure image 205B may be in the form of an image file (e.g., with a BMP, TIFF, LJPEG, or PNG, among others).

The pre-procedure image 205A may identify or have at least one region of interest (ROI) 230A (also referred herein as a structure of interest (SOI), a volume of interest (VOI), or feature of interest (FOI)). The post-procedure image 205B may identify or have at least ROI 230B. Each ROI 230A and 230B may correspond to an area, a section, or a portion of the respective pre-procedure image 205A and post-procedure image 205B correlated or associated with a feature within the lung region 220 of the subject 225. The feature may be, for example, a tumor within the lung region 220 when the pre-procedure image 205A is acquired, a tissue portion where the tumor was present in the lung region 220 when the post-procedure image 205B is acquired. The feature may also be, for instance, an anatomical structure within the lung region 220 in both the pre-procedure image 205A and the post-procedure image 205B, such as a bronchi, bronchioles, alveoli, pleura, or blood vessels, among others. The ROI 230A and 230B may correspond to a contiguous portion (e.g., as depicted) or one or more non-contiguous portions within the respective pre-procedure image 205A and post-procedure image 205B.

The parameter set 210 may identify or define an administration of the ablation procedure to the lung region 220 of the subject 225. The ablation procedure may have been administered to the subject 225 via at least one applicator (e.g., a probe, a needle, or electrode) to apply or feed energy onto a portion of within the lung region 220 to remove or destroy tumorous cells of the lung cancer. The ablation procedure may be radiofrequency ablation (RFA) to apply high-frequency radio waves; microwave ablation (MWA) to apply a thermal energy in the microwave portion of the electromagnetic field; cryoablation to apply extreme cold; laser ablation using a laser; or a high-intensity focused ultrasound (HIFU) to direct ultrasound energy onto the tumor, among others. In some embodiments, the parameter set 210 may have been created, inputted, or generated by a clinician examining the subject 225 or administering the ablation procedure to the lung region 220. In some embodiments, the parameter set 210 may have been automatically generated or determined from the applicator (or a computing device coupled with the applicator) used to deliver the ablation procedure the lung region 220.

The parameter set 210 may include, for example: a position of an applicator within the lung region 220 (defined within the pre-procedure image 205A or the post-procedure image 205B using pixel coordinates); a size of the applicator itself (e.g., defined using pixel coordinates or physical measurement of the applicator device); an amount of power applied (e.g., defined in terms of Watts); a tip location of the applicator (e.g., defined using pixel coordinates) from which the power is emitted; a duration of the application of the power (e.g., ranging from 30 seconds to 30 minutes); an interval of time between applications (e.g., 1 to 15 minute intervals between each application); an orientation of the applicator within the lung region 220 when applying the energy to the tumor; a trajectory of the applicator through the lung region 220 (defined within the pre-procedure image 205A or the post-procedure image 205B using pixel coordinates); and a type of ablation procedure (e.g., RFA, MWA, cryoablation, laser, or HIFU), among others.

The annotation 215 may identify or include information about the pre-procedure image 205A, the post-procedure image 205B, and the parameter set 210, among others. The annotation 215 may define, label, or otherwise identify the ROI 230A within the pre-procedure image 205A and the ROI 230B within the post-procedure image 205B. For example, the annotation 215 may identify the ROI 230A and the ROI 230B within the pre-procedure image 205A and the post-procedure image 205B using pixel coordinates. In some embodiments, the ROI 230A and 230B may have been manually created, inputted, or generated by a clinician examining the pre-procedure image 205A and the post-procedure image 205B respectively. In some embodiments, the ROI 230A and 230B may have been automatically generated using an image segmentation model applied (e.g., by the model trainer 125) to the pre-procedure image 205A and the post-procedure image 205B.

Continuing on, the annotation 215 may include or identify at least one ablation region 235. In some embodiments, the annotation 215 may identify or include an expected mask identifying or defining the ablation region 235. The ablation region 235 may define or identify a portion, an area, or a segment within the post-procedure image 205B (or the pre-procedure image 205A) associated with or corresponding to the administration of the ablation procedure within the lung region 220 of the subject 225. For instance, the ablation region 235 may correspond to a volume of the energy (e.g., thermal energy for MWA) emitted by the applicator when delivering the ablation procedure to the tumor. The ablation region 235 may be defined in terms of pixel coordinates within the post-procedure image 205B (or the pre-procedure image 205A). The ablation region 235 may have been manually created, inputted, or generated by a clinician administering the ablation procedure to the tumor in the lung region 220 of the subject 225. The ablation region 235 of the annotation 215 may not correspond to any anatomical boundary (e.g., exterior, bronchi, bronchioles, alveoli, pleura, or blood vessels) associated with the lung region 220 of the subject 225. In some embodiments, at least a portion of the annotation 215 may be part of the parameter set 210, and vice-versa.

The image preparer 135 executing on the image processing system 105 may execute, carry out, or otherwise perform registration of the pre-procedure image 205A with the post-procedure image 205B, using features within the images, such as the ROI 230A and the ROI 230B. The image registration may be, for example, in accordance with deformable image registration, intensity-based registration, landmark-based registration, surface-based registrations, B-spline registration, and atlas-based registration, or any combination thereof, among others. The image registration may take into account the anatomical or structural features within the pre-procedure image 205A and the post-procedure image 205B.

To perform the registration, the image preparer 135 may determine or identify the features within the pre-procedure image 205A with the post-procedure image 205B (e.g., the ROI 230A and 230B respectively). Based on the identified features, the image preparer 135 may calculate or determine at least one correspondence between the pre-procedure image 205A and the post-procedure image 205B. For example, the image preparer 135 may determine an alignment of the features (e.g., ROI 230A and 230B) between the pre-procedure image 205A and the post-procedure image 205B. The correspondence may define a spatial relationship (e.g., defined using pixels) between the features to align the feature in the pre-procedure image 205A with the feature in the post-procedure image 205B. In determining the correspondence, the image preparer 135 may perform an optimization for the alignment of the pre-procedure image 205A with the post-procedure image 205B. For instance, the image preparer 135 may minimize the discrepancy between the features in the pre-procedure image 205A with the feature in the post-procedure image 205B using a deformation estimation field in accordance with deformable image registration.

In some embodiments, the image preparer 135 may alter, control, or otherwise constrain the registration of the pre-procedure image 205A with the post-procedure image 205B. The constraint may be a function of a feature of the pre-procedure image 205A (e.g., an anatomical structure corresponding to ROI 230A) and a feature of the post-procedure image 205B (e.g., an anatomical structure corresponding to ROI 230B). The function may also factor in the administration of the ablation procedure, using the parameter set 210 and other assumption or information regarding the ablation procedure. The constraint may be to prevent or reduce large deformations between the pre-procedure image 205A with the post-procedure image 205B. For example, the constraint may limit the alignment transformations for correspondences in features between the two images in areas about the ROIs 230A and 230B (e.g., tumor or other anatomical structures), or ablation region 235.

Using the correspondence between the features in the pre-procedure image 205A and the post-procedure image 205B, the image preparer 135 may output, produce, or otherwise generate at least one registered image 205C. The registered image 205C may be a modification of the pre-procedure image 205A based on the alignment transformations for the correspondence between the features (e.g., ROIs 230A and 230B) in the pre-procedure image 205A and the post-procedure image 205B. The registered image 205C may be used to train the ablation prediction model 150 to predict ablation zones from images acquired prior to the administration of the ablation on the lung region. In some embodiments, the image preparer 135 may limit the modification to a portion of the pre-procedure image 205A in generating the registered image 205C. For instance, the image preparer 135 may warp, alter, or otherwise modify a portion of the pre-procedure image 205A (e.g., about the ROI 230A) in accordance with the correspondence to align with or fit with the post-procedure image 205B.

In some embodiments, the image preparer 135 may perform the image registration between the pre-procedure image 205A and the post-procedure image 205B in accordance with a sequence of image registration operations. In general, the sequence (or hierarchy) of image registration operations may align, correspond, or otherwise register the pre-procedure image 205A and the post-procedure image 205 at different levels, starting from the overall image, the overall lungs, individual anatomical structures, and then to ROIs (e.g., tumors or ablation zones), among others. The sequence may include one or more of: (1) an image registration between the pre-procedure image 205A and the post-procedure image 205B; (2) an image registration between the pre-procedure image 205A and the post-procedure image 205B (e.g., between the overall images); (3) an image registration between a portion of the pre-procedure image 205A corresponding to the lung region 220 and a portion of the post-procedure image 205B corresponding to the lung region 220 (e.g., between the lungs in each biomedical image); (4) an image registration between the ROI 230A of the pre-procedure image 205A and the ROI 230B of the post-procedure image 205B, or (5) an image registration between a portion of the pre-procedure image 205A corresponding to an anatomical structure in the lung region 220 and a portion of the post-procedure image 205B corresponding to a corresponding anatomical structure in the lung region 220. In some embodiments, the image registration operations may be in a different sequence. For example, the fifth registration operation may be performed prior to the second through fourth image registration operations.

First, the image preparer 135 may perform the image registration between the pre-procedure image 205A and the post-procedure image 205B. The image registration may be an affine registration between the overall pre-procedure image 205A and the overall post-procedure image 205B. The affine registration may be performed by the image preparer 135 to align (e.g., coarsely, approximately, or roughly) the overall pre-procedure image 205A with the overall post-procedure image 205B. The alignment may include, for example, rotation, scaling, shearing, or translation of the pre-procedure image 205A with the post-procedure image 205B, or vice-versa. In performing the affine image registration, the image preparer 135 may identify or determine alignment between a portion of the pre-procedure image 205A corresponding to an anatomical structure with a corresponding portion of the post-procedure image 205B corresponding to the same anatomical structure. The anatomical structure may include, for example, an exterior of the lobe, a bronchial tree, or alveoli, among others. Using the alignment, the image preparer 135 may change, modify, or otherwise transform the pre-procedure image 205A with the post-procedure image 205B, or vice versa.

Second, the image preparer 135 may perform the image registration between the pre-procedure image 205A and the post-procedure image 205B. The image registration may be a deformable image registration to account for motion (e.g., due to contraction or expansion from breathing) or other perturbances of the overall imaging across the pre-procedure image 205A and the post-procedure image 205B. The deformable image registration may be used to find correspondences including distortions of features, besides rotation, scaling, shearing, or translation. In some embodiments, the deformable image registration may use multiple resolutions (e.g., using subsampling techniques including grid-based, random, or pyramidal based approaches, such as Gaussian pyramids). In performing the deformable registration, the image preparer 135 may identify or determine the correspondences (e.g., defined via B-splines or free-form deformations (FFD)) between the overall pre-procedure image 205A and the post-procedure image 205B. Using the correspondences, the image preparer 135 may change, modify, or otherwise transform the pre-procedure image 205A to register with the post-procedure image 205B, or vice versa.

Third, the image preparer 135 may perform the image registration between a portion of the pre-procedure image 205A associated with the lung region 220 and a portion of the post-procedure image 205B associated with the lung region 220. The image registration may be a deformable image registration to account for motion (e.g., due to contraction or expansion from breathing) or other perturbances of lung region 220 across the pre-procedure image 205A and the post-procedure image 205B. In some embodiments, the deformable image registration may use multiple resolutions (e.g., using subsampling techniques including grid-based, random, or pyramidal based approaches, such as Gaussian pyramids). In performing the deformable image registration, the image preparer 135 may detect, identify, or otherwise determine correspondences (e.g., defined via B-splines or free-form deformations (FFD)) between the overall lung (e.g., exterior edge of the lobe lung or lung parenchyma) in the pre-procedure image 205A with the post-procedure image 205B. Using the correspondences, the image preparer 135 may transform the pre-procedure image 205A to register with the post-procedure image 205B, or vice versa.

Fourth, the image preparer 135 may perform the image registration between the ROI 230A of the pre-procedure image 205A and the ROI 230B of the post-procedure image 205B. The image registration may be a deformable image registration to account for motion or other perturbances of the ROIs 230A and 230B across the across the pre-procedure image 205A and the post-procedure image 205B. In some embodiments, the deformable image registration may use multiple resolutions (e.g., using subsampling techniques including grid-based, random, or pyramidal based approaches, such as Gaussian pyramids). In performing the deformable image registration, the image preparer 135 may detect, identify, or otherwise determine correspondences (e.g., defined via B-splines or free-form deformations (FFD)) between the ROI 230A of the pre-procedure image 205A with the ROI 230B of the post-procedure image 205B. Using the correspondences, the image preparer 135 may change, modify, or otherwise transform the pre-procedure image 205A to register with the post-procedure image 205B, or vice versa.

Fifth, the image preparer 135 may perform the image registration between a portion of the pre-procedure image 205A corresponding to an anatomical structure in the lung region 220 and a portion of the post-procedure image 205B corresponding to a corresponding anatomical structure in the lung region 220. The image registration may be an landmark-based image registration to match a portion of the pre-procedure image 205A corresponding to an anatomical structure in the lung region 220 with a portion of the post-procedure image 205B corresponding to a corresponding anatomical structure in the lung region 220. The anatomical structure may include any feature within the lung region 220, such as alveoli, alveolar ducts, alveolar sac, bronchioles, terminal bronchioles, respiratory bronchioles, alveolar pores, tumors, blood vessels (e.g., pulmonary capillaries, pulmonary arteries, pulmonary veins, bronchial arteries, bronchial veins, lymphatic vessels, subsegmental arteries and veins, and any branch points), and calcifications, among others. In performing the image registration, the image preparer 135 may

In some embodiments, in performing the registration operations, the image preparer 135 may use one or more registration parameters. The parameters may include, for example: masking (e.g., a segmentation mask for features in the images), image similarity metric (e.g., mutual information or cross correlation), optimization function (e.g., stochastic gradient descent) sampling (e.g., sub-sampling for image resolutions), image interpolation (e.g., tri-linear interpolation), non-rigid or deformable transformations (e.g., defined using B-splines or free form deformation model (FFD) to manipulate shape of features in images), regularization (e.g., penalty for abrupt variations), multi-resolution, rigidity value (e.g., enforce non-rigid registration on certain anatomies), and optimization cost (e.g., similarity metric, bending energy regularization, rigidity value, etc.), among others. Certain parameters may be used in particular image registration operations in the sequence. For instance, the parameters for non-rigid or deformable registration may be used by the image preparer 135 when performing the deformable image registration operations. The sequence of image registration operations and the registration parameters may be as detailed herein in Example 3.

The parameter analyzer 140 executing on the image processing system 105 may calculate, determine, or otherwise generate at least one application region 240 within the registered image 205C using the parameter set 210. In some embodiments, the parameter analyzer 140 may generate the application region 140 within the pre-procedure image 205A, and invoke the image preparer 135 to modify the application region 140 to be defined within the registered image 205C using the correspondence determined from the image registration. The application region 240 may define or identify a portion of the registered image 205C corresponding to application of the ablation procedure within the lung region 220. For instance, the application region 240 may define the portion of the registered image 205C corresponding to a volume within the lung region 225 administered with the ablation treatment. The application region 240 may also correspond to the portion of the registered image 205C defining the energy emitted by the applicator when delivering the ablation treatment.

To generate, the parameter analyzer 140 may extract, select, or otherwise identify one or more parameters from the parameter set 210. The identified parameters may include the position, the size, the orientation, the power, the tip location, and the trajectory of the applicator within the lung region 220, among others. Using the parameters, the parameter analyzer 140 may identify or determine a corresponding portion within the registered image 205C for the application region 240. For example, the parameter analyzer 140 may map the tip location of the applicator to the pixel coordinates within the registered image 205C. From these coordinates, the parameter analyzer 140 may use the orientation and the power delivered by the applicator to determine the application region 240. The mapping may factor in the correspondences from the image registration.

In some embodiments, the parameter analyzer 140 may transform, change, or otherwise convert the registered image 205C and the parameter set 210 from an original coordinate system to a target coordinate system (also referred herein as an application-centric coordinate system). The target coordinate system may be defined relative to the applicator to administer the ablation procedure within the lung region 220 of the subject 225. The target coordinate system may be defined relative to the tip position of the applicator. For example, the target coordinate system may be a local ellipsoidal system with the center at the tip position of the applicator, and may be used to define an ellipsoid corresponding to the energy emitted by the applicator when administering the ablation procedure. The target coordinate system may be used to compare measured and predicted ablation regions across images from different subjects.

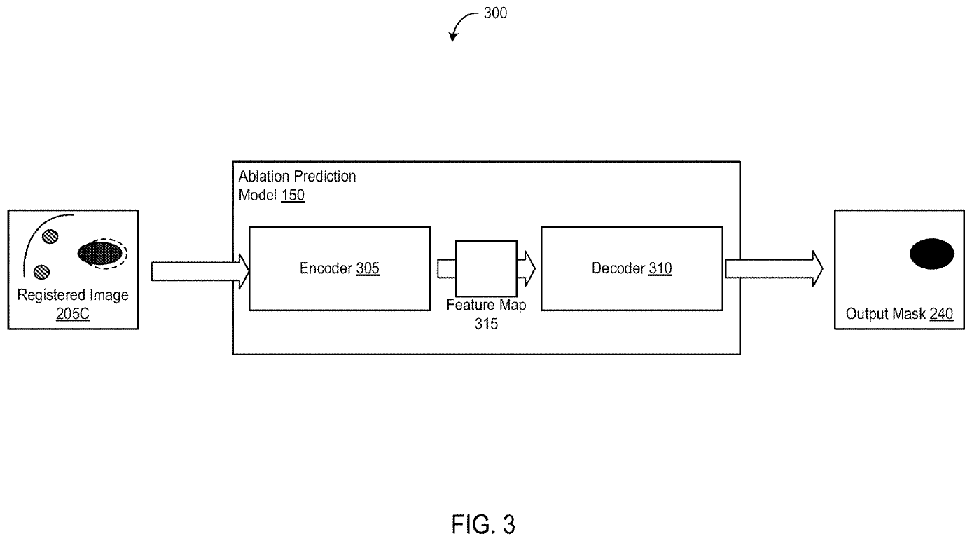

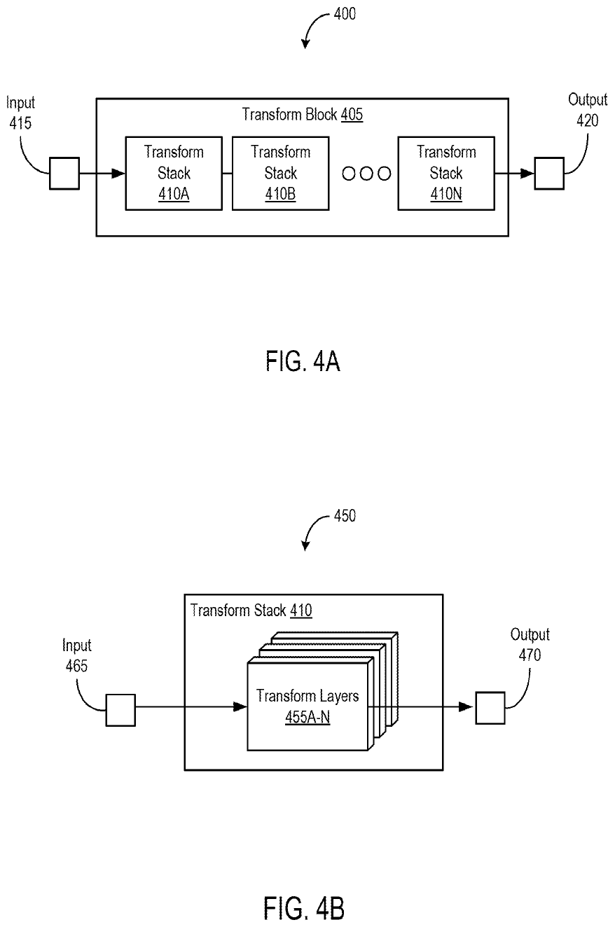

Moving onto FIG. 2B, the model trainer 125 may initialize and establish the ablation prediction model 150. The ablation prediction model 150 may have a set of weights (sometimes herein referred to as kernel parameters, kernel weights, or parameters) to determine predicted ablation regions in biomedical images (e.g., of lung regions in subjects with lung cancer). The set of weights may be arranged in a set of transform layers with one or more connections with one another to relate inputs and outputs of the ablation prediction model 150. In some embodiments, the ablation prediction model 150 may be implemented using the U-net architecture as described herein in Examples 1 and 2. In some embodiments, the ablation prediction model 150 may be implemented using the architecture as detailed herein in conjunction with FIGS. 3-4B. In initializing, the model trainer 125 may calculate, determine, or otherwise generate the initial values to assign the set of weights of the ablation prediction model 150 using pseudo-random values or fixed defined values.

With the identification of the example from the training dataset 160, the model applier 130 executing on the image processing system 105 may feed or apply the registered image 205C and the corresponding parameter set 210 of each example in the training dataset 160 to the ablation prediction model 150. The corresponding parameter set 210 may be associated with the same example identifying the pre-procedure image 205A and the post-procedure image 205B used to derive or generate the registered image 205C. In some embodiments, the model applier 130 may apply the registered image 205C with the application region 240 to the ablation prediction model 150. In feeding, the model applier 130 may process the registered image 205C and the parameter set 210 in accordance with the set of weights of the ablation prediction model 150. In some embodiments, the model applier 130 may carry out, execute, or otherwise perform one or more pre-processing functions on the registered image 205C, prior to application to the ablation prediction model 150. For example, the model applier 130 may alter or re-size the dimensions of the registered image 205C to dimensions compatible with the input of the ablation prediction model 150.

From processing using the weights of the ablation prediction model 150, the model applier 130 may produce, create, or otherwise generate at least one output mask 240. The output mask 240 may be similar to the pre-procedure image 205A, the post-procedure image 205B, or the registered image 205C (e.g., in dimensions or modality). The output mask 240 may define, identify, or otherwise include at least one predicted ablation region 235′ within the registered image 205C or by extension the post-procedure image 205B. The predicted ablation region 235′ may at least partially define or identify a segment, portion, or an area within the post-procedure image 205B (or the pre-procedure image 205A or the registered image 205C) corresponding to the administration of the ablation procedure. For example, the predicted ablation region 235′ may correspond to a predicted volume of the energy (e.g., thermal energy for MWA) emitted by the applicator when delivering the ablation procedure to the tumor in the lung region 225 of the subject 225. The model applier 130 may traverse through the selected examples in the training dataset 160 and apply the registered image 205C and the parameter set 210 from each example to the ablation prediction model 150.

With the generation, the model trainer 125 may calculate, generate, or otherwise determine at least one loss metric 245 to be used to modify, adjust, or otherwise update the weights of the ablation prediction model 150. The loss metric 245 may correspond to a degree of deviation between the ablation region 235 identified in the annotation 215 and the predicted ablation region 235′ from the output mask 240. To determine, the model trainer 125 may compare the predicted ablation region 235′ from the ablation prediction model 150 with the ablation region 235 as identified in the annotation 215. In some embodiments, the model trainer 125 may compare the output mask 240 identifying the predicted ablation region 235′ with the expected mask defining the ablation region 235 of the annotation 215. The comparison may be a pixel-by-pixel comparison.

From comparing, the model trainer 125 may determine the loss metric 245 to indicate the degree of deviation. The loss metric 245 may be generated using any number of loss functions, such as a norm loss (e.g., L1 or L2), mean absolute error (MAE), mean squared error (MSE), a quadratic loss, a cross-entropy loss, and a Huber loss, among others. In some embodiments, the model trainer 125 may determine a similarity metric as a function of the expected mask and the output mask 240. The similarity metric may be used as the loss metric 245, and the function used to calculate the similarity metric may include, for example, a Dice co-efficient metric, a Jaccard index, or an overlap coefficient, among others. In some embodiments, the model trainer 125 may determine the loss metric 245 based on a combination of the loss function resultant (e.g., cross-entropy loss) and the similarity metric (e.g., Dice score).

Using the loss metric 245, the model trainer 125 may modify, change, or otherwise update at least one weights of the ablation prediction model 150. The updating of the weights may be in accordance with backpropagation algorithm and may include dropping units and connections within the ablation prediction model 150. From updating, the model trainer 125 may encode the ablation prediction model 150 to generate output masks 240 to identify the predicted ablation regions 235′ more accurately and precisely. The updating of weights of the ablation prediction model 150 may be in accordance with an optimization function (also referred herein as an objective function). The optimization function may define one or more rates or parameters at which the weights of the ablation prediction model 150 are to be updated. The optimization function may be in accordance with stochastic gradient descent, and may include, for example, an adaptive moment estimation (Adam), implicit update (ISGD), and adaptive gradient algorithm (AdaGrad), among others. The updating of the weights of the ablation prediction model 150 may be repeated until convergence. Upon completion of training, the model trainer 125 may store and maintain the set of weights of the ablation prediction model 150 on the database 155 to be used to generate output masks from newly acquired images.

Referring now to FIG. 3, depicted is a block diagram of an architecture 300 for the ablation prediction model 150 in the system 100 for determining ablation regions. Under the architecture 300, the ablation prediction model 150 may include at least one encoder 305 and at least one decoder 310, among others. The set of weights of the ablation prediction model 150 may be configured, arrayed, or otherwise arranged across the encoder 305 and the decoder 310 of the ablation prediction model 150. The inputs and outputs of encoder 305 and the decoder 310 may be connected in any configuration, such as in series (e.g., as depicted), in parallel, or any combination thereof. The architecture of the encoder 305 and the decoder 310 for the architecture 300 is detailed herein below in conjunction with FIGS. 4A and 4B.

The ablation prediction model 150 may have at least one input and at least one output. The input and output may be related to one another via the set of weights arranged across the encoder 305 and the decoder 310. The input for the ablation prediction model 150 may include at least one registered image 205C and may correspond to an input of the encoder 305. In the ablation prediction model 150, the encoder 305 may generate at least one feature map 315 using the input registered image 205C. The feature map 315 may be a lower dimensional representation of the corresponding input registered image 205C in a latent feature space. The output of the encoder 305 may be fed forward to the decoder 310. Using the feature map 315 from the encoder 305, the decoder 310 in turn may generate the output mask 240. The output mask 240 may correspond to output for the ablation prediction model 150 and may identify the predicted ablation region 235′.

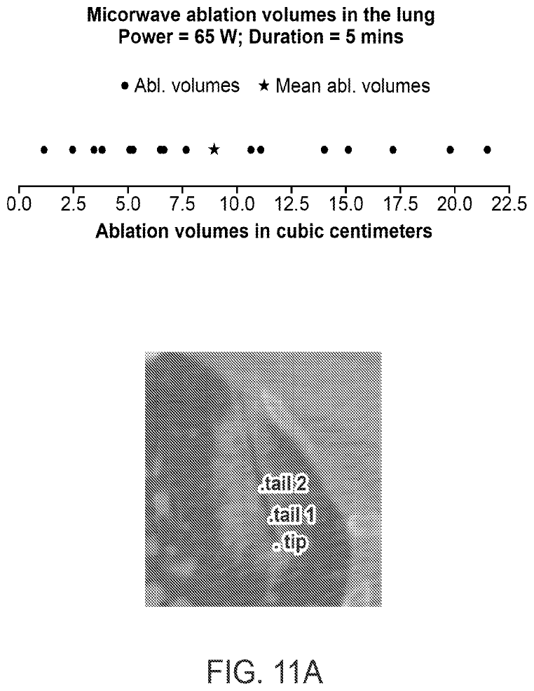

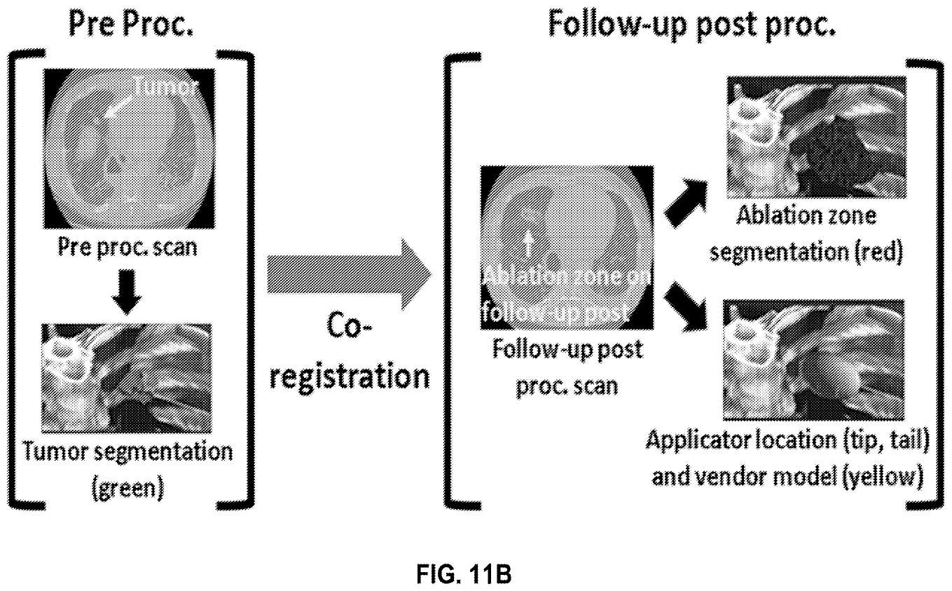

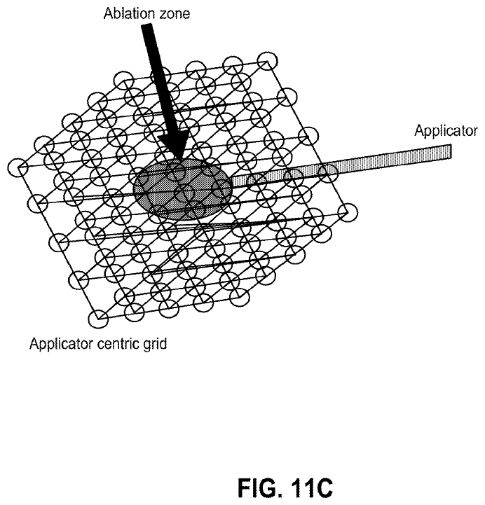

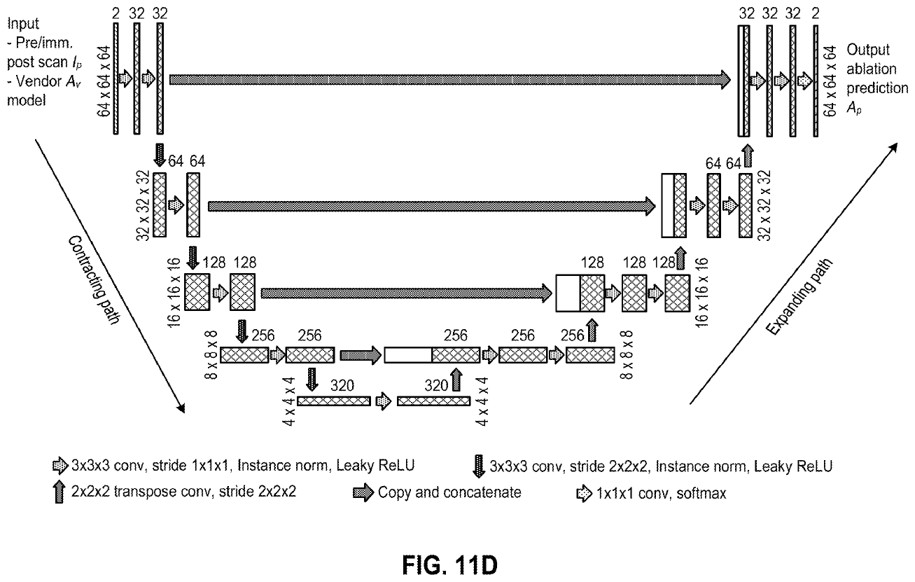

Referring now to FIG. 4A, depicted is a block diagram of an architecture 400 of a transform block in the ablation prediction model 150 in the system 100 for determining ablation regions. The transform block 405 may be used to implement the encoder 305 and the decoder 310 in the ablation prediction model 150. For example, the encoder 305 and the decoder 310 may each be an instance of the transform block 405. Under the architecture 400, the transform block 405 may include one or more transform stacks 410A-N (hereinafter generally referred to as a transform stack 410). The set of transform stacks 410 can be arranged in series (e.g., as depicted) or parallel configuration, or in any combination. The set of transform stacks 410, for example, may be arranged as shown in FIG. 11D in accordance with the U-net like architecture. In a series configuration, the input of one transform stack 410 may include the output of the previous transform stack 410 (e.g., as depicted). In parallel configuration, the input of one transform stack 410 may include the input of the entire transform block 405.

The transform block 405 may include at least one input 415 and at least one output 420. The set of weights of the encoder 305 or the decoder 310 may be arranged across the transform stacks 410 may define the relationship between the input 415 and the output 420. When used to implement the encoder 305, the input 415 may correspond to the registered image 205C, and the output 420 may be the feature map 315. When used to implement the decoder 310, the input 415 may include the feature map 315 generated by the encoder 305, and the output 420 may be the output mask 420 for the overall ablation prediction model 150. The size of the transform stacks 410 may vary throughout the transform block 405. For example, the transform stacks 410 of the encoder 305 may decrease from 224×224 to 14×14. Conversely, the transform stacks 410 of the decoder 310 may increase from 256×1 to 224×224.

Referring now to FIG. 4B, depicted is a block diagram of an architecture of a set of transform layers in a transform block in an ablation prediction model in the system 100 for determining ablation regions. The transform stack 410 may be used to implement the encoder 305 and the decoder 310. Under the architecture 450, the transform stack 410 may include a set of transform layers 455A-N (hereinafter generally referred to as transform layers 455). The transform stack 410 may include at least one input 465 and at least one output 470. The input 465 and the output 470 may be related to each other via the set of kernel parameters defined across the transform layers 455. The set of transform layers 455 can be arranged in any configuration such as in series or in parallel, or any combination thereof. For example, under series configuration, the transform layers 455 may have an output of one transform layer 455 fed as an input to a succeeding transform layer 455.

Each transform layer 455 may have a non-linear input-to-output characteristic. The transform layer 455 may comprise a convolutional layer, a normalization layer, and an activation layer (e.g., a rectified linear unit (ReLU)), among others. When used to implement the encoder 305, the transform layers 455 of the transform stack 410 may be configured or arranged as a convolutional neural network (CNN) or a transformer neural network. For example, the convolutional layer, the normalization layer, and the activation layer (e.g., a softmax function, sigmoid non-linearity function, or rectified linear unit (ReLU)) in the transform layers 455 may be arranged in accordance with a fully convolutional neural network (FCNN). When used to implement the encoder 305, the transform layer 455 may include at least one pooling or down-sampling operator layer. When used to implement the decoder 310, the transform layer 455 may include at least one up-sampling operator layer.

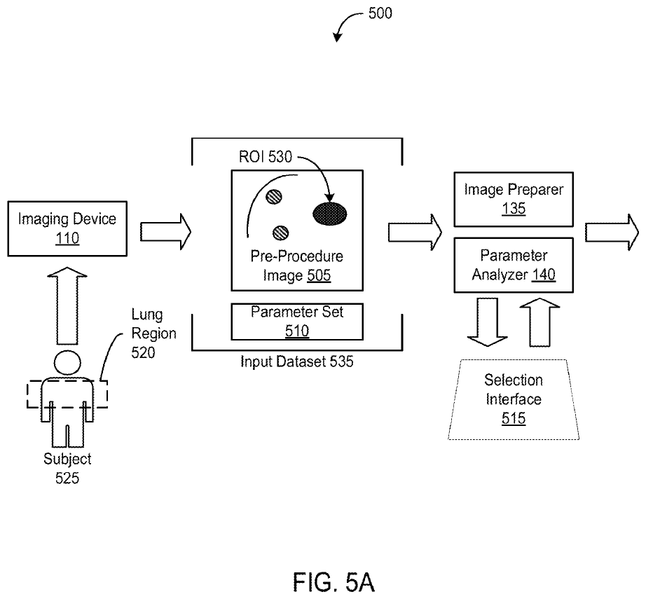

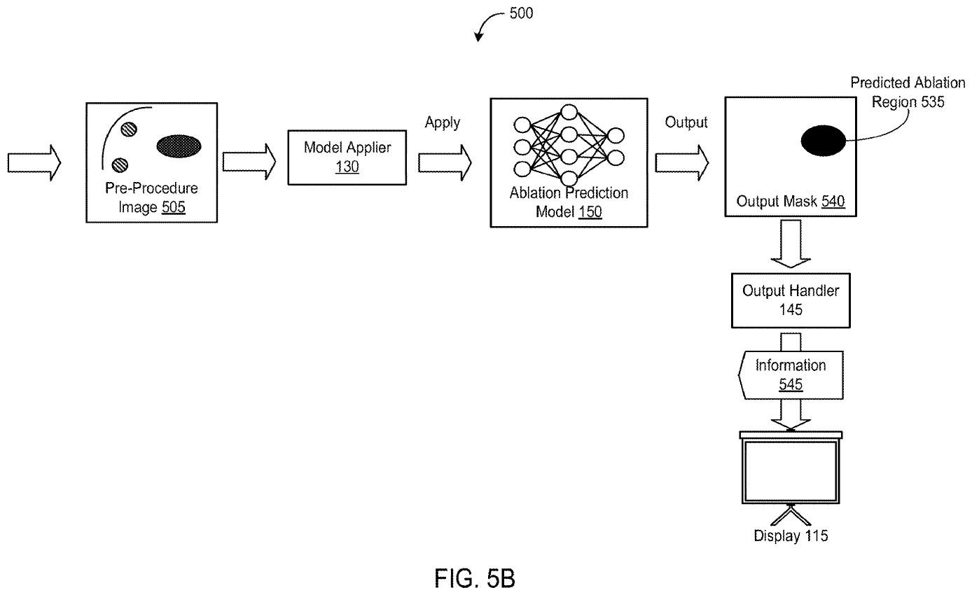

Referring now to FIGS. 5A and 5B, depicted are block diagrams of a process 500 for applying an ablation prediction model to acquired biomedical images in the system 100 for determining ablation regions. The process 500 may include or correspond to operations performed in the system 100 for applying the ablation prediction model 150 to newly acquired images. The operations of the process 500 may be similar to one or more of the operations in the process 200 as discussed above. Starting with FIG. 5A, under the process 500, the imaging device 110 may output, produce, otherwise generate at least one pre-procedure image 505. The pre-procedure image 505 may be similar (e.g., in modality or dimensions) to the pre-procedure image 205A respectively. In some embodiments, the imaging device 110 may generate the pre-procedure image 505, without acquiring or generating a post-procedure image, to diagnose and plan for the ablation procedure prior to the administration of the procedure. In some embodiments, the imaging device 110 may acquire and generate a post-procedure image (e.g., in a similar manner as the post-procedure image 205B) to evaluate along with the pre-procedure image 505.

The imaging device 110 may scan, obtain, or otherwise acquire the pre-procedure image 505. The pre-procedure image may be derived, acquired, or otherwise be of at least a portion the lung region 520 of the subject 525 (e.g., a human patient or an animal). The lung region 520 may generally correspond to a portion or a volume of the subject 525 in which at least one lung is located. For example, the lung region 520 may correspond a chest cavity of the subject 525 in which one or both pair of lungs are located. Both the pre-procedure image 505 may capture at least one tumor associated with the incidence of lung cancer within the lung region 520. The pre-procedure image 505 may be acquired prior to the administration of the ablation procedure (e.g., a day to a month before the procedure). The post-procedure image may be acquired subsequent to the administration of the ablation procedure (e.g., a week to one or two months after the procedure).

The pre-procedure image 505 may be acquired using any number of imaging modalities in accordance with lung cancer screening techniques. For example, each of the pre-procedure image 505 may include an X-ray scan, a computed tomography (CT) scan, a computed tomography laser mammography (CTLM), a magnetic resonance imaging (MRI) scan, a nuclear magnetic resonance (NMR) scan, an ultrasound imaging scan, a positron emission tomography (PET) scan, or a photoacoustic spectroscopy scan, among others, of the lung region 520 of the subject 525. The pre-procedure image 505 may include a set of two-dimensional cross-sections (e.g., a front, a sagittal, a transverse, or an oblique plane) acquired from the three-dimensional volume. The pre-procedure image 505 may be defined in terms of pixels, in two-dimensions or three-dimensions. Although primarily discussed in terms of CT scans, other imaging modalities besides those listed above may be supported by the image processing system 105 for the pre-procedure image 505. The pre-procedure image 505 may be in the form of an image file (e.g., with a BMP, TIFF, LJPEG, or PNG, among others).

The pre-procedure image 505 may identify or have at least one region of interest (ROI) 530 (also referred herein as a structure of interest (SOI), a volume of interest (VOI), or feature of interest (FOI)). The ROI 530 may correspond to an area, a section, or a portion of the respective pre-procedure image 505 correlated or associated with a feature within the lung region 520 of the subject 525. The feature may be, for example, a tumor within the lung region 520 when the pre-procedure image 505 is acquired. The feature may also be, for instance, an anatomical structure within the lung region 520 in both the pre-procedure image 505, such as a bronchi, bronchioles, alveoli, pleura, or blood vessels, among others. The ROI 530 may correspond to a contiguous portion (e.g., as depicted) or one or more non-contiguous portions within the respective pre-procedure image 505.

The ROI 530 may be identified be identified within the pre-procedure image 505. In some embodiments, the ROI 530 may have been manually created, inputted, or generated by a clinician examining the pre-procedure image 505. In some embodiments, the ROI 530 may have been automatically generated using an image segmentation model applied (e.g., by the model trainer 125) to the pre-procedure image 505.

The imaging device 110 may output, create, or otherwise generate at least one parameter set 510. In some embodiments, another computing device communicatively coupled with the image processing system 105 may generate the parameter set 510. The parameter set 510 may identify or define an administration of the ablation procedure to the lung region 520 of the subject 525. The ablation procedure may have been administered to the subject 525 via at least one applicator (e.g., a probe, a needle, or electrode) to apply or feed energy onto a portion of within the lung region 520 to remove or destroy tumorous cells of the lung cancer. The ablation procedure may be radiofrequency ablation (RFA) to apply high-frequency radio waves; microwave ablation (MWA) to apply a thermal energy in the microwave portion of the electromagnetic field; cryoablation to apply extreme cold; laser ablation using a laser; or a high-intensity focused ultrasound (HIFU) to direct ultrasound energy onto the tumor, among others. In some embodiments, the parameter set 510 may have been created, inputted, or generated by a clinician examining the subject 525 or administering the ablation procedure to the lung region 520. In some embodiments, the parameter set 510 may have been automatically generated or determined from the applicator (or a computing device coupled with the applicator) used to deliver the ablation procedure the lung region 520.

The parameter set 510 may include, for example: a position of an applicator within the lung region 520 (defined within the pre-procedure image 505 using pixel coordinates); a size of the applicator itself (e.g., defined using pixel coordinates or physical measurement of the applicator device); an amount of power applied (e.g., defined in terms of Watts); a tip location of the applicator (e.g., defined using pixel coordinates) from which the power is emitted; a duration of the application of the power (e.g., ranging from 30 seconds to 30 minutes); an interval of time between applications (e.g., 1 to 15 minute intervals between each application); an orientation of the applicator within the lung region 520 when applying the energy to the tumor; a trajectory of the applicator through the lung region 520 (defined within the pre-procedure image 505 using pixel coordinates); and a type of ablation procedure (e.g., RFA, MWA, cryoablation, laser, or HIFU), among others.

Upon acquisition, the imaging device 110 (or a computing device coupled thereto) may provide, send, or otherwise transmit at least one input dataset 535 to the image processing system 105. The input dataset 535 may include data for administration of ablation procedure to the lung region 520 of the corresponding subject 525. The input dataset 535 may identify or include: the pre-procedure image 505, and the parameter set 510. In some embodiments, the input dataset 535 may lack the post-procedure image and the parameter set 510. The input dataset 535 may be provided with metadata including, for example, an anonymized identifier for the subject 525, a timestamp of acquisition of the input dataset 535, and device manufacturer information for the imaging device 110, among others.

In some embodiment, the imaging device 110 may send the input dataset 535 for storage and maintenance on a database (e.g., the database 155) for subsequent processing by the image processing system 105. In some embodiments, the imaging device 110 may provide the input dataset 535 to the image processing system 105 in response to a request to process the input dataset 535. The request may be inputted by a clinician examining the subject 525 to diagnose and evaluate the effect of ablation procedure on the lung region 520 of the subject 525. He request may also be inputted by the clinician prior to the administration of the ablation procedure to determine which parameters to set for the subject 525. In some embodiments, the imaging device 110 may provide the pre-procedure image 505 to the image processing system 105, without the post-procedure image or the parameter set 510 when the request is prior the administration of the ablation procedure.

The image preparer 135 may retrieve, receive, or otherwise identify the input dataset 535 from the imaging device 110. From the input dataset 535, the image preparer 135 may extract, obtain, or identify the pre-procedure image 505. In some embodiments, the image preparer 135 may identify the pre-procedure image 505, without the post-procedure image and the parameter set 510 when the input dataset 535 lacks the post-procedure image and the parameter set 510. The image preparer 135 may determine whether to perform image registration based on the contents of the input dataset 535. If the input dataset 535 includes both the pre-procedure image 505 and the post-procedure image, the image preparer 135 may determine to proceed with an image registration. Otherwise, if the input dataset 535 includes the pre-procedure image 505 and not the post-procedure image, the image preparer 135 may determine to forego the image registration.

With the identification, the image preparer 135 may execute, carry out, or otherwise perform registration of the pre-procedure image 505 with the post-procedure image, using features within the images, such as the ROI 530 and an ROI within the post-procedure image. The image registration may be, for example, in accordance with deformable image registration, intensity-based registration, landmark-based registration, surface-based registrations, B-spline registration, and atlas-based registration, or any combination thereof, among others. The image registration may take into account the anatomical or structural features within the pre-procedure image 505 and the post-procedure image. In some embodiments, the image preparer 135 may skip or omit the registration of images, for example, when the input dataset 535 lacks any post-procedural images.

To perform the registration, the image preparer 135 may determine or identify the features within the pre-procedure image 505 with the post-procedure image (e.g., the ROIs respectively). Based on the identified features, the image preparer 135 may calculate or determine at least one correspondence between the pre-procedure image 505 and the post-procedure image. The correspondence may define a spatial relationship (e.g., defined using pixels) between the features to align the feature in the pre-procedure image 505 with the feature in the post-procedure image. In determining the correspondence, the image preparer 135 may perform an optimization for the alignment of the pre-procedure image 505 with the post-procedure image.

In some embodiments, the image preparer 135 may alter, control, or otherwise constrain the registration of the pre-procedure image 505 with the post-procedure image. The constraint may be a function of a feature of the pre-procedure image 505 (e.g., an anatomical structure corresponding to ROI 530) and a feature of the post-procedure image (e.g., an anatomical structure corresponding to the ROI). The function may also factor in the administration of the ablation procedure, using the parameter set 510 and other assumption or information regarding the ablation procedure. The constraint may be to prevent or reduce large deformations between the pre-procedure image 505 with the post-procedure image.

Using the correspondence between the features in the pre-procedure image 505 and the post-procedure image, the image preparer 135 may output, produce, or otherwise generate at least one registered image. The registered image may be a modification of the pre-procedure image 505 based on the alignment transformations for the correspondence between the features (e.g., ROIs) in the pre-procedure image 505 and the post-procedure image. In some embodiments, the image preparer 135 may limit the modification to a portion of the pre-procedure image 505 in generating the registered image. For instance, the image preparer 135 may warp, alter, or otherwise modify a portion of the pre-procedure image 505 (e.g., about the ROI 530) in accordance with the correspondence to align with or fit with the post-procedure image.

The parameter analyzer 140 may extract, obtain, or otherwise identify the parameter set 510 from the input dataset 535. In some embodiments, the parameter analyzer 140 may provide a set of candidate parameter sets 510 for selection via a selection interface 515. For example, the parameter analyzer 140 may provide the selection interface 515 (e.g., a graphical user interface) to a computing device from which to select one or more of the candidate parameter sets 510 for planning the administration of the ablation procedure to deliver to the subject 525. A user (e.g., a clinician) of the computing device can use the interface to select one or more of the candidate parameter sets 510 to evaluate the predicted effect of the administration of the ablation procedure on the lung region 520 as depicted in the pre-procedure image 505, prior to the performance of the ablation procedure. Each of the candidate parameter sets 510 may have different parameters, such as varying position of the applicator, size, amount of power, duration, interval, orientation, duration, and procedure type, among others. The selection interface 515 may be used to input, assign, or set values for the parameters of the candidate parameter sets 510. The parameter analyzer 140 may retrieve, identify, or receive the selection of the parameter set 510 from the candidate parameter sets 510 via the selection interface 515.

The parameter analyzer 140 may calculate, determine, or otherwise generate at least one application region 540 within the pre-procedure image 505 using the parameter set 510. In some embodiments, the parameter analyzer 140 may generate the application region 540 within the registered image. In some embodiments, the parameter analyzer 140 may generate the application region 140 within the pre-procedure image 505, and invoke the image preparer 135 to modify the application region 140 to be defined within the pre-procedure image 505 using the correspondence determined from the image registration. The application region 540 may define or identify a portion of the pre-procedure image 505 corresponding to application of the ablation procedure within the lung region 520. For instance, the application region 540 may define the portion of the pre-procedure image 505 corresponding to a volume within the lung region 525 administered with the ablation treatment. The application region 540 may also correspond to the portion of the pre-procedure image 505 defining the energy emitted by the applicator when delivering the ablation treatment.

To generate, the parameter analyzer 140 may extract, select, or otherwise identify one or more parameters from the parameter set 510. The identified parameters may include the position, the size, the orientation, the power, the tip location, and the trajectory of the applicator within the lung region 520, among others. Using the parameters, the parameter analyzer 140 may identify or determine a corresponding portion within the pre-procedure image 505 for the application region 540. For example, the parameter analyzer 140 may map the tip location of the applicator to the pixel coordinates within the pre-procedure image 505. From these coordinates, the parameter analyzer 140 may use the orientation and the power delivered by the applicator to determine the application region 540. The mapping may factor in the correspondences from the image registration.

In some embodiments, the parameter analyzer 140 may transform, change, or otherwise convert the pre-procedure image 505 and the parameter set 510 from an original coordinate system to a target coordinate system (also referred herein as an application-centric coordinate system). The target coordinate system may be defined relative to the applicator to administer the ablation procedure within the lung region 520 of the subject 525. The target coordinate system may be defined relative to the tip position of the applicator. For example, the target coordinate system may be a local ellipsoidal system with the center at the tip position of the applicator, and may be used to define an ellipsoid corresponding to the energy emitted by the applicator when administering the ablation procedure. The target coordinate system may be used to compare measured and predicted ablation regions across images from different subjects.

Moving onto FIG. 5B, the model applier 130 may feed or apply the pre-procedure image 505 and the corresponding parameter set 510 of each example in the training dataset 160 to the ablation prediction model 150. The corresponding parameter set 510 may be associated with the pre-procedure image 505. In some embodiments, the model applier 130 may apply the registered image with the application region 540 to the ablation prediction model 150. In some embodiments, the model applier 130 may apply the pre-procedure image 505 with the parameter set 510 (or the application region 540), when the input dataset 535 lacks the post-procedure image. In feeding, the model applier 130 may process the pre-procedure image 505 and the parameter set 510 in accordance with the set of weights of the ablation prediction model 150. In some embodiments, the model applier 130 may carry out, execute, or otherwise perform one or more pre-processing functions on the pre-procedure image 505, prior to application to the ablation prediction model 150. For example, the model applier 130 may alter or re-size the dimensions of the pre-procedure image 505 to dimensions compatible with the input of the ablation prediction model 150.

From processing using the weights of the ablation prediction model 150, the model applier 130 may produce, create, or otherwise generate at least one output mask 540. The output mask 540 may be similar to the pre-procedure image 505 (e.g., in dimensions or modality). The output mask 540 may define, identify, or otherwise include at least one predicted ablation region 535 within the pre-procedure image 505. When generated from the pre-procedure image 505, the output mask 540 may define the predicted ablation region 535 within the pre-procedure image 505. The predicted ablation region 535 may at least partially define or identify a segment, portion, or an area within the pre-procedure image 505 (or the registered image) corresponding to the administration of the ablation procedure.

The output handler 145 executing on the image processing system 105 may store and maintain an association between the subject 525 (e.g., using an identifier) and the predicted ablation region 535, using one or more data structures (e.g., arrays, matrixes, tables, linked lists, stacks, queues, trees, or heaps). In some embodiments, the output handler 145 may generate the association among two or more of the subject 525, the pre-procedure image 505, the parameter set 510, the predicted ablation region 535, and the output mask 540, among others. In some embodiments, the output handler 145 may generate the association with the post-procedure image and the registered image, when the post-procedure image is also received. Upon generation, the output handler 145 may store the association onto the database 155.

Based on the association between the subject 525 and the predicted ablation region 535, the output handler 145 may create, determine, or otherwise generate information 545. The information 545 may include or identify any one or more of the following: the identifier for the subject 525, the pre-procedure image 505, the parameter set 510, the predicted ablation region 535, and the output mask 540, among others. With the generation, the output handler 145 may send, transmit, or otherwise provide the information 545 to the display 115. The provision of the information 545 may be in response to a request from a user (e.g., clinician examining the subject 525 for the ablation procedure administered or to be administered to a tumor in the lung region 525). In some embodiments, the output handler 145 may provide the information 545 for the performance of the ablation procedure in accordance with the predicted ablation region 535.

The display 115 (or a computing device connected thereto) may display, render, or otherwise present the information 545 from the image processing system 105. The information 545 may be presented to the clinician examining the subject 525 to evaluate the treatment of the lung cancer. For example, when used to evaluate a previously administered ablation treatment, the display 115 may present the pre-procedure image 505, which is overlaid with an outline corresponding to the predicted ablation region 535. The information 545 may be presented to the clinician examining the subject 525 to plan out the administration of the ablation procedure within the lung region 525. For instance, when used to diagnose and plan the administration, the display 115 may render the pre-procedure image 505 overlaid with the predicted ablation region 535, along with an option of different parameter sets 510 defining the ablation procedure to be performed. By selecting different parameter sets 510, the clinician can evaluate the effect of each of the different parameter sets 510 in delivering the ablation procedure in the lung region 520 of the subject 525. In some embodiments, the clinician can perform the ablation procedure in accordance with the predicted ablation region 535 identified in the information 545. In some embodiments, the clinician can plan the ablation procedure in accordance with the predicted ablation region 535 identified in the information 545. For example, the clinician may use the predicted ablation region 535 to generate a plan for the ablation procedure. According to the plan, the clinician may use an applicator to perform the ablation procedure 545 to the affected region within the lung region 520 of the subject 525.

In this manner, the image processing system 105 may use image registration and machine learning (ML) techniques to quickly and accurately assess the effects of ablation procedures to treat lung cancer in subjects in need thereof. This may lower or eliminate the involvement of manual and subjective examination of scans of lung regions 520 from subjects 525, thus reducing the time and effort spent by clinicians in providing such assessments. Furthermore, the use of the image registration techniques during training in particular may encode the weights of the ablation prediction model 150 to predict ablation zones more accurately and precisely from newly acquired pre-procedure images. With higher accuracy and precision, the image processing system 105 may be able to lower consumption of computing resource and network bandwidth that would have been otherwise spent in manually and repeatedly creating ablation procedure plans. Furthermore, since the predicted ablation zones determined by the ablation prediction model 150 of the image processing system 105 may be used to generate or guide the creation of ablation procedure plans, the clinical outcome for the subject 525 in treating cancer or tumors in the lung region 520 can be improved.

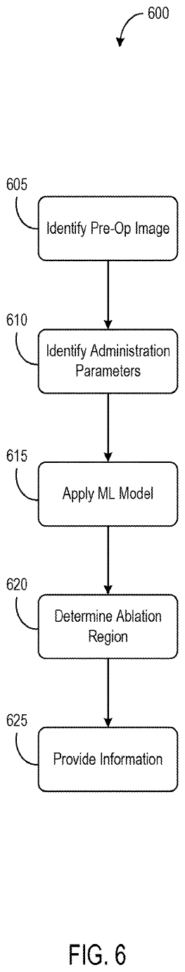

Referring now to FIG. 6, depicted is a flow diagram of a method 600 of determining ablation regions from biomedical images of subjects. The method 600 may be implemented using or performed by any of the components described herein, such as the image processing system 105 in conjunction with FIG. 1-5 or the system 800 in FIG. 8. Under the method 600, a computing system (e.g., the image processing system 105) may identify a pre-operation image (e.g., the pre-procedure image 505A) (605). The computing system may identify ablation administration parameters (e.g., the parameter set 510) (610). The computing system may apply a machine learning (ML) model (e.g., the ablation prediction model 150) to the pre-operation image and the ablation parameters (615). From applying the ML model, the computing system may determine an ablation region (e.g., the predicted ablation region 535′) (620). The computing system may provide information (e.g., the information 545) (625).

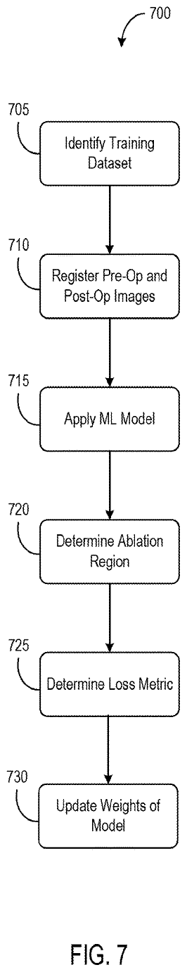

Referring now to FIG. 7, depicted is a flow diagram of a method 700 of training models to determine ablation regions from biomedical images of subjects. The method 700 may be implemented using or performed by any of the components described herein, such as the image processing system 105 in conjunction with FIG. 1-5 or the system 800 in FIG. 8. Under the method 700, a computing system (e.g., the image processing system 105) may identify a training dataset (e.g., the training dataset 160) including a pre-operation image (e.g., the pre-procedure image 205A), a post-operation image (the post-procedure image 205B), a set of ablation parameters (e.g., the parameter set 210), and an annotation (e.g., the annotation 215) (705). The computing system may register the pre-operation image with the post-operation image (710). The computing system may apply a machine learning (ML) model (e.g., the ablation prediction model 150) to a registered image (e.g., the registered image 205C) and the ablation parameters (715). From applying the ML model, the computing system may determine an ablation region (e.g., the predicted ablation region 235′) (720). The computing system may determine a loss metric (e.g., the loss metric 245) (725). The computing system may update weights of the ML model (730).

### Network and Computing Environments

Various operations described herein can be implemented on computer systems. FIG. 8 shows a simplified block diagram of a representative server system 800, client computing system 814, and network 826 usable to implement certain embodiments of the present disclosure. In various embodiments, server system 800 or similar systems can implement services or servers described herein or portions thereof. Client computing system 814 or similar systems can implement clients described herein. Server system 800 can have a modular design that incorporates a number of modules 802 (e.g., blades in a blade server embodiment); while two modules 802 are shown, any number can be provided. Each module 802 can include processing unit(s) 804 and local storage 806.

Processing unit(s) 804 can include a single processor, which can have one or more cores, or multiple processors. In some embodiments, processing unit(s) 804 can include a general-purpose primary processor as well as one or more special-purpose co-processors such as graphics processors, digital signal processors, or the like. In some embodiments, some or all processing unit(s) 804 can be implemented using customized circuits, such as application specific integrated circuits (ASICs) or field programmable gate arrays (FPGAs). In some embodiments, such integrated circuits execute instructions that are stored on the circuit itself. In other embodiments, processing unit(s) 804 can execute instructions stored in local storage 806. Any type of processors in any combination can be included in processing unit(s) 804.

Local storage 806 can include volatile storage media (e.g., DRAM, SRAM, SDRAM, or the like) and/or non-volatile storage media (e.g., magnetic or optical disk, flash memory, or the like). Storage media incorporated in local storage 806 can be fixed, removable, or upgradeable as desired. Local storage 806 can be physically or logically divided into various subunits such as a system memory, a read-only memory (ROM), and a permanent storage device. The system memory can be a read-and-write memory device or a volatile read-and-write memory, such as dynamic random-access memory. The system memory can store some or all of the instructions and data that processing unit(s) 804 need at runtime. The ROM can store static data and instructions that are needed by processing unit(s) 804. The permanent storage device can be a non-volatile read-and-write memory device that can store instructions and data even when module 802 is powered down. The term “storage medium” as used herein includes any medium in which data can be stored indefinitely (subject to overwriting, electrical disturbance, power loss, or the like) and does not include carrier waves and transitory electronic signals propagating wirelessly or over wired connections.

In some embodiments, local storage 806 can store one or more software programs to be executed by processing unit(s) 804, such as an operating system and/or programs implementing various server functions such as functions of the systems 100 or any other system described herein, or any other server(s) associated with systems 100 or any other system described herein.

“Software” refers generally to sequences of instructions that, when executed by processing unit(s) 804, cause server system 800 (or portions thereof) to perform various operations, thus, defining one or more specific machine embodiments that execute and perform the operations of the software programs. The instructions can be stored as firmware residing in read-only memory and/or program code stored in non-volatile storage media that can be read into volatile working memory for execution by processing unit(s) 804. Software can be implemented as a single program or a collection of separate programs or program modules that interact as desired. From local storage 806 (or non-local storage described below), processing unit(s) 804 can retrieve program instructions to execute and data to process in order to execute various operations described above.

In some server systems 800, multiple modules 802 can be interconnected via a bus or other interconnect 808, forming a local area network that supports communication between modules 802 and other components of server system 800. Interconnect 808 can be implemented using various technologies including server racks, hubs, routers, etc.

A wide area network (WAN) interface 810 can provide data communication capability between the local area network (interconnect 808) and the network 826, such as the Internet. Technologies can be used, including wired (e.g., Ethernet, IEEE 802.3 standards) and/or wireless technologies (e.g., Wi-Fi, IEEE 802.11 standards).

In some embodiments, local storage 806 is intended to provide working memory for processing unit(s) 804, providing fast access to programs and/or data to be processed while reducing traffic on interconnect 808. Storage for larger quantities of data can be provided on the local area network by one or more mass storage subsystems 812 that can be connected to interconnect 808. Mass storage subsystem 812 can be based on magnetic, optical, semiconductor, or other data storage media. Direct attached storage, storage area networks, network-attached storage, and the like can be used. Any data stores or other collections of data described herein as being produced, consumed, or maintained by a service or server can be stored in mass storage subsystem 812. In some embodiments, additional data storage resources may be accessible via WAN interface 810 (potentially with increased latency).

Server system 800 can operate in response to requests received via WAN interface 810. For example, one of modules 802 can implement a supervisory function and assign discrete tasks to other modules 802 in response to received requests. Work allocation techniques can be used. As requests are processed, results can be returned to the requester via WAN interface 810. Such operation can generally be automated. Further, in some embodiments, WAN interface 810 can connect multiple server systems 800 to each other, providing scalable systems capable of managing high volumes of activity. Other techniques for managing server systems and server farms (collections of server systems that cooperate) can be used, including dynamic resource allocation and reallocation.

Server system 800 can interact with various user-owned or user-operated devices via a wide area network such as the Internet. An example of a user-operated device is shown in FIG. 8 as client computing system 814. Client computing system 814 can be implemented, for example, as a consumer device such as a smartphone, other mobile phone, tablet computer, wearable computing device (e.g., smart watch, eyeglasses), desktop computer, laptop computer, and so on.

For example, client computing system 814 can communicate via WAN interface 810. Client computing system 814 can include computer components such as processing unit(s) 816, storage device 818, network interface 820, user input device 822, and user output device 837. Client computing system 814 can be a computing device implemented in a variety of form factors, such as a desktop computer, laptop computer, tablet computer, smartphone, other mobile computing device, wearable computing device, or the like.

Processing unit(s) 816 and storage device 818 can be similar to processing unit(s) 804 and local storage 806 described above. Suitable devices can be selected based on the demands to be placed on client computing system 814; for example, client computing system 814 can be implemented as a “thin” client with limited processing capability or as a high-powered computing device. Client computing system 814 can be provisioned with program code executable by processing unit(s) 816 to enable various interactions with server system 800.

Network interface 820 can provide a connection to the network 826, such as a wide area network (e.g., the Internet) to which WAN interface 810 of server system 800 is also connected. In various embodiments, network interface 820 can include a wired interface (e.g., Ethernet) and/or a wireless interface implementing various RF data communication standards such as Wi-Fi, Bluetooth, or cellular data network standards (e.g., 3G, 4G, LTE, etc.).

User input device 822 can include any device (or devices) via which a user can provide signals to client computing system 814; client computing system 814 can interpret the signals as indicative of particular user requests or information. In various embodiments, user input device 822 can include any or all of a keyboard, touch pad, touch screen, mouse or other pointing device, scroll wheel, click wheel, dial, button, switch, keypad, microphone, and so on.

User output device 837 can include any device via which client computing system 814 can provide information to a user. For example, user output device 837 can include display-to-display images generated by or delivered to client computing system 814. The display can incorporate various image generation technologies, e.g., a liquid crystal display (LCD), light-emitting diode (LED) including organic light-emitting diodes (OLED), projection system, cathode ray tube (CRT), or the like, together with supporting electronics (e.g., digital-to-analog or analog-to-digital converters, signal processors, or the like). Some embodiments can include a device such as a touchscreen that function as both input and output device. In some embodiments, other user output devices 837 can be provided in addition to or instead of a display. Examples include indicator lights, speakers, tactile “display” devices, printers, and so on.

Some embodiments include electronic components, such as microprocessors, storage and memory that store computer program instructions in a computer readable storage medium. Many of the features described in this specification can be implemented as processes that are specified as a set of program instructions encoded on a computer readable storage medium. When these program instructions are executed by one or more processing units, they cause the processing unit(s) to perform various operations indicated in the program instructions. Examples of program instructions or computer code include machine code, such as is produced by a compiler, and files including higher-level code that are executed by a computer, an electronic component, or a microprocessor using an interpreter. Through suitable programming, processing unit(s) 804 and 816 can provide various functionality for server system 800 and client computing system 814, including any of the functionality described herein as being performed by a server or client, or other functionality.

It will be appreciated that server system 800 and client computing system 814 are illustrative and that variations and modifications are possible. Computer systems used in connection with embodiments of the present disclosure can have other capabilities not specifically described here. Further, while server system 800 and client computing system 814 are described with reference to particular blocks, it is to be understood that these blocks are defined for convenience of description and are not intended to imply a particular physical arrangement of component parts. For instance, different blocks can be but need not be located in the same facility, in the same server rack, or on the same motherboard. Further, the blocks need not correspond to physically distinct components. Blocks can be configured to perform various operations, e.g., by programming a processor or providing appropriate control circuitry, and various blocks might or might not be reconfigurable depending on how the initial configuration is obtained. Embodiments of the present disclosure can be realized in a variety of apparatus including electronic devices implemented using any combination of circuitry and software.

### EXAMPLES

The present technology is further illustrated by the following Examples, which should not be construed as limiting in any way.

### Example 1: Experimental Methods

### Work-Flow of the Methods

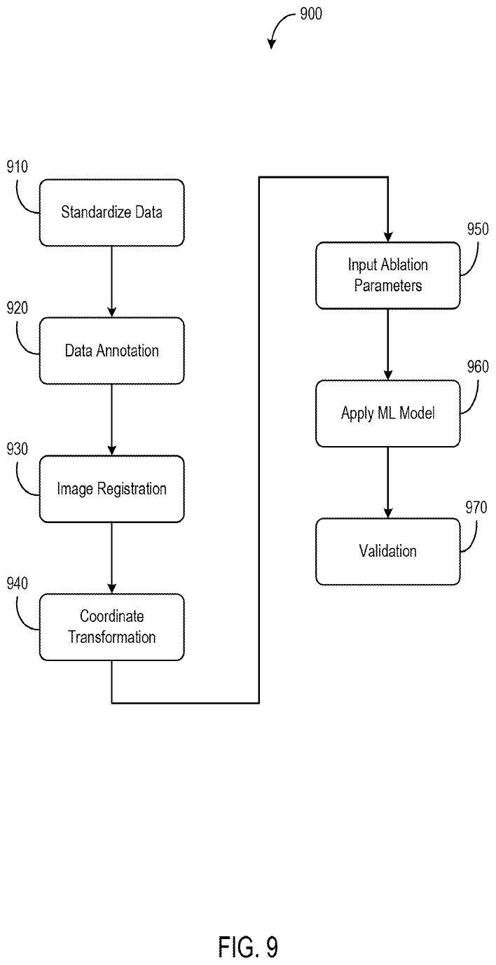

Referring now to FIG. 9, depicted is a flow diagram of a work flow 900 for predicting ablation zones in lung computed tomography (CT) scans. The process or work flow 900 may be implemented using any of the components described herein, such as the image processing system 105 as detailed above.

### 1. Data Standardization of Multiple Samples in Database

At step 910, data standardization refers to steps to standardize image intensities of the scans obtained from different scanners with different image acquisition parameters, image reconstruction algorithms, image quality and resolution, and variations across patients. This may include low-level image intensity manipulations such as normalization to a consistent intensity range, bias and variance corrections, thresholding etc. Such operations may be done on per scan basis or using intensity characteristics across a population of scans.

### 2. Annotating the Data in Each Sample-Segmentation, Needle Coordinates

At step 920, data annotation refers to steps to define the ground truth labels and to allow for image registration for training/testing. Tumor and lung ablation segmentations were performed manually by a radiologist. The applicator coordinates were defined by a radio-lucent shadow visible on the follow-up scan. A radiologist confirmed these coordinates by comparison with the intra-procedure imaging. Segmentations and applicator coordinate demarcations were performed using 3D Slicer.

### 3. Performing Registration Step on Each Sample for Training the Algorithm

At step 930, this step registers the pre-procedure scan with the follow-up post-procedure scans, establishing the relationship between the image intensities and anatomy in the scans for AI training. Typical image registration procedures using whole scans are inadequate for ablation workflows because of the significant non-rigid motion of the lung, complex motion at the lung-chest wall interface, distortions/displacements that occur during applicator insertion, differences in patient positioning during ablation and tissue contraction that occurs as a result of ablation. We present a novel, multi-stage deformable registration procedure that is specific to the ablation workflow.

### 4. Performing Coordinate Transformation on Each Sample

At step 940, to compare ablation zones across patients at different locations and orientations, we define a co-ordinate system that is intrinsic to the ablation zone, which we call the application-centric coordinate system (ACCS). This is a 3D rectangular grid of voxel positions aligned with the applicator and centered at the applicator tip. Each CT scan and each segmentation mask undergoes this coordinate transformation.

### 5. Inputting Ablation Parameters

At step 950, the ablation parameters (applicator position, power, duration) were incorporated as a second input channel in the model (the first input channel being the pre-procedure patient scan). We gathered the manufacturer data from ex vivo porcine lung ablations using the applicator vendor's planning software. These provide expected dimensions of ablation ellipsoids for power ranging from 35 W to 65 W at 5 W intervals and from 1 to 10 minutes at 1 minute intervals. We use linear interpolation to obtain expected dimensions for power and duration in between these settings. We construct ellipsoids from these dimensions with 1 s inside the ellipsoid and 0 s elsewhere. The ellipsoid is then transformed into the ACCS coordinate system.

### 6. DNN Machine Learning Step Performed on a Training Set Cohort

At step 960, a deep neural network (DNN) model is trained to predict the expected ablation zone (output) from the pre-procedure CT and ablation parameters (input). While in our specific experiments, we employed a U-Net architecture based model via the nnUNet framework, more generally, any DNN capable of dense prediction can be designed to predict the output.

7. Difference from Anatomy Segmentation

Although we use a neural network architecture commonly used for segmentation of anatomical structures, we emphasize this task is completely different than density estimation/segmentation. Namely, there are no clearly discernible boundaries on the pre-procedure scan that define the ablation zone unlike typical image segmentation where the object of interest has a clear boundary. There is only the original tumor and normal lung anatomy. Although we are using a model that is commonly used for segmentation, this is not a segmentation task, hence is a much harder problem.

### 8. Validation

At step 970, we perform cross-validation to estimate the generalizability of our algorithm to predict the ablation zone extent and shape.

### Data

The data was obtained as part of an institutional review board-approved retrospective study of patients who underwent MWA at our institution between January 2015 and January 2019. Exclusion criteria included use of a probe other than NeuWave PR (Ethicon US, Raritan, NJ), multiple probes or multiple burns performed at the same site, two or more adjacent sites with overlapping ablation zones, or if background lung parenchyma could not be differentiated from the ablation zone. These criteria resulted in 113 unique ablations from 72 patients (see Table 1). Pre-procedure and one month follow-up post-procedure CT scans were processed. MWA power and duration were obtained from radiology reports.

TABLE 1
Participant characteristics.

| | | | |
|---|---|---|---|
| Number of | Median (IQR) | | Number of |
| participants | age (years) | Gender split | ablations |

Initial data distribution

| | | | |
|---|---|---|---|
| 72 | 57 (49-67) | Women: 41 | 113 |
| | | Men: 31 | |

Data distribution used for training (TRE <=2 mm)

| | | | |
|---|---|---|---|
| 40 | 56 (49-65) | Women: 20 | 52 |
| | | Men: 20 | |

### Pre-Processing Pipeline

FIGS. 11B-11C provide an overview of the pre-processing steps in our pipeline. The tumor and ablation zone were manually segmented in the pre-procedure and follow-up scans, respectively (FIG. 11B), by a radiologist with >15 years of experience. Applicator position was defined by the radio-lucent shadow visible on the follow-up scan (FIG. 11C: top). All segmentations and applicator coordinate demarcations were performed using 3D Slicer software (v4.11.1).¹³

For the vendor model, we used the vendor's user interface software (v3.1.0), which provided dimensions of expected ablation ellipsoids for different power (35 W to 65 W at 5 W increments) and durations (one to 10 minutes at one minute increments). Ablation zone dimensions were linearly interpolated for power and duration in between these specifications.

We applied a deformable image registration procedure, utilizing the free-form deformation model based on B-spline transformation¹⁴ to register the pre-procedure and follow-up scans (FIG. 11B). An initial rigid registration was performed for coarse alignment, followed by a coarse-to-fine multi-resolution deformable registration wherein the result of each resolution acted as input to the next.¹⁵ We constrained the registration by the relevant regions of interest surrounding the tumor and ablation zone and applied a rigidity penalty on the tumor to avoid large deformations. The registration optimization used mutual information to measure similarity between images and restricted excessive bending of the transform to avoid unnatural deformations. We followed Klein et al. to set various registration parameters.¹⁶ All registrations were performed using the SimpleElastix¹⁷ image registration library (v0.9.1).¹⁸

To compare ablation zones across patients, we defined an applicator-centric coordinate system (ACCS),¹⁹ a 3D rectangular grid of voxel positions aligned with the applicator and centered at the applicator tip (see FIG. 11C, bottom). The ACCS grid size was set to 64 mm³ and sampling rate of 1 mm. Numpy (v1.21.2), SciPy (v1.7.3), and NiBabel (v3.2.1) python libraries were used for data processing.

### Model

Our model was a fully convolutional neural network based on the U-Net²⁰ architecture (FIG. 11D). It consisted of an encoder that took the 3D pre-procedure scan and the binary vendor ellipsoid as input channels, and a decoder that produced the predicted ablation zone as output. The segmented ablation zones on the follow-up scans were used as ground truth. The encoder/decoder, comprised of five levels, consisted of convolution, downsampling, non-linearity, and upsampling operations (data not shown).²¹⁻²³

### Training

We utilized the sum of cross entropy and Dice coefficient between the predicted and ground truth ablation zone to train the network.²⁰ The data was split into seven train-test cross-validation (CV) folds and training and testing were carried out on all the seven folds independently. In each train-test fold, the training data was further split into five train-validation CV folds, with 80% of the training data used for training and 20% used for validation in each fold. The purpose of these further CV splits was to train different models on each of the five CV subsets of the training data whose predictions on the corresponding test set were ensembled by averaging their softmax outputs. As a result, we obtained five different models from each train-test CV split, resulting in a total of 7×5=35 models on the whole data set. We followed Isensee et al. for the training methodology (data not shown).^{20,24}

### Statistics

Image registrations were evaluated using the target registration error (TRE).²⁵ Wilcoxon signed-rank test was used to compare vendor to sample distributions for volume and shape statistics (sphericity, elongation, and surface-to-volume ratio [SVR]). Volumes and shapes of the predicted and vendor model from true ablation zones were compared using Bland-Altman plots.²⁶ Paired and two sample t-tests were used to test for differences from the ground truth and vendor model. Levene's test was used to test the differences in the limits of agreement (LOA).²⁷ The degree of overlap between prediction and ground truth was computed using the Dice coefficient,²⁸ precision and recall. The mean and 95% confidence intervals²⁹ were computed to obtain summaries of the performance of our algorithm, which were compared against that of the vendor model. Statsmodel (v0.13.5) and SciPy (v1.7.3) python libraries were used for all analysis.

### Registration

The registration method is based on a two-stage process: 1) in the first stage, we perform an initial rigid registration that ensures the images are globally aligned and provides a good initialization for the subsequent deformable registration; and 2) in the second stage, we perform multi-resolution deformable image registration to account for the non-rigid motion of the lung anatomy to obtain a finer match [Klein S, et al (2009) IEEE Trans Med Imaging 29:196-205; Lester H, Arridge S R (1999) Pattern Recognit 32:129-149]. We additionally incorporate various constraints optimized for ablation workflows that aid in achieving successful registrations. Below we describe the various parameters of the registration.

### Parameters Used for Registration:

Focusing on the region of interest (ROI): We are only interested in registering the anatomy close to the regions of interest, i.e., tumor in pre scan and ablation zone in follow-up post scan. The ablation prediction is not affected by anatomical features far away. E.g., the ablation zone in the left lung is not directly influenced by the blood vessels in the right lung. We achieve this by first 1) cropping the images in 3D using spherical masks of chosen radii (e.g., 70 mm), centered at the segmentations. This results in spherical images with the tumor/ablation zone at the center, removing any effect of far-away structures as well as speeding up registrations since the cropped images are smaller; and more importantly using 2) registration masks to constrain the registration to be driven by only the intensities inside a further smaller spherical region centered on the tumor/ablation zone (e.g., 30-50 mm radius). These ensures the immediately surrounding anatomy of tumor/ablation zone has the most impact on the registration, increasing the likelihood of accurate registrations in these regions. We remove the tumor and the ablation zone segmentations themselves from these spherical masks to ensure they do not contribute to the registration process. This is done to avoid the non-rigid transform from attempting to match the tumor with the bigger ablation zone by expanding/altering its size and shape.
Image similarity metric: image mutual information [Viola P, Wells III W M (1997) Int J Comput Vis 24:137-154] the number of intensity histogram bins for computing the joint distribution was chosen to be 32.
Optimizer: stochastic gradient descent, where “step size” was adapted as a function of the similarity between the gradient directions in the current and the previous iterations [Klein S, Pluim J P W, Staring M, Viergever M A (2009) Int J Comput Vis 81:227-239]. The maximum number of iterations was set to 256.
Image sampler: image intensities were sampled from 2048 locations (including sub-voxel) from the image regions inside the specified registration masks, with new spatial samples selected in every iteration.
Image interpolation: We used tri-linear interpolation during the registration. For computing the final transformed image, B-spline based cubic interpolation is used for better quality.
Initial rigid registration: The images are first aligned using a simpler rigid transformation before proceeding to the more complex non-rigid deformations. This ensures the images are globally aligned first and provides a good initialization for the subsequent non-rigid registrations [Klein S, et al (2009) IEEE Trans Med Imaging 29:196-205; Lester H, Arridge SR (1999) Pattern Recognit 32:129-149]. The rigid transformation was composed of a 3D rotation matrix and a translation vector. The centroids of tumor in pre and ablation zone in follow-up post are also matched with a certain weight in the loss function to guide the initial alignment.
Non-rigid/deformable registration: We utilize the free form deformation model (FFD) based on B-splines [Rueckert D, Sonoda L I, Hayes C, et al (1999) IEEE Trans Med Imaging 18:712-721] as the transformation function. FFDs are defined by a grid of control points which can be used to manipulate the shape of 3D objects. Using cubic B-splines, a hyperpatch is defined by the control points in 3D that is smooth and continuous. Moving the control points of the FFD deforms the B-spline transformation function, which in turn results in smooth deformations of the underlying fixed image pixel grid. The parameters of the transform are the positions of the control points, which are optimized during the registration to maximize the similarity between the deformed moving and fixed images.
Regularization: We utilized the bending energy as our regularizer [Rueckert D, Sonoda L I, Hayes C, et al (1999) IEEE Trans Med Imaging 18:712-721; Wahba G (1990) Spline models for observational data. SIAM], which penalizes abrupt variations in the transformation (e.g., high expansion followed by high compression) and can avoid folding.
Multi-resolution strategies: A coarse-to-fine hierarchical image registration at multiple resolutions was performed, where images were smoothed using specified Gaussian kernels to different resolutions and registered incrementally starting with the lowest [Lester H, Arridge SR (1999) Pattern Recognit 32:129-149]. The lowest resolutions reduce the number of local optima, increasing the likelihood of finding the global optima. This strategy aligns large structures first, with the finer details introduced and matched at successively higher resolutions. The number of resolutions was chosen to be 4, with the registration proceeding from the lower to the higher resolutions in succession. The lower resolution images were obtained by smoothing the image with a Gaussian of σ=0.5 in the finest resolution and increased by a factor of 2 in each successive lower resolution. A similar strategy is followed for the B-spline transformation, where the complexity of the transform (i.e., the number of individual parameters in the transform) is increased from low to high during successive registrations at increasingly higher resolution levels [Klein S, et al (2009) IEEE Trans Med Imaging 29:196-205; Rueckert D, Sonoda L I, Hayes C, et al (1999) IEEE Trans Med Imaging 18:712-721]. At lower image resolutions, a coarse B-spline control point grid is used with higher spacing between control points; this allows for only coarse deformations, aiding to match larger global structures. Whereas at higher resolutions, a finer grid is used with lesser spacing between the control points; this allows for local deformations, aiding to match smaller local structures in the images. The control point grid spacing started with 8 mm along all dimensions at the finest resolution and was increased to 11.28, 15.84, and 22.4 mm in successive lower resolutions.
Rigidity penalty on tumor region: While non-rigid registration matches match the anatomy surrounding the tumor in the pre and ablation zone in the follow-up post scans, we desire the tumor area itself to not undergo large deformations. Preserving volume and shape morphology of the tumor is to learn a meaningful relationship with the ablation zone. We achieve this by imposing a rigidity penalty on the tumor region in the pre scan following Staring et al. [Staring M, Klein S, Pluim J P W (2007) Med Phys 34:4098-4108]. The specific constraints include: 1) Affinity condition: This condition ensures that the second order partial derivatives of the transform with respect to the spatial coordinates is 0, as required by a rigid transform (and more generally an affine transform); 2) Orthonormality condition: This condition enforces the property that the rotation matrix should be orthonormal; and 3) Properness: This condition ensures that the determinant of the rotation matrix is 1. This is equivalent to imposing the determinant of the Jacobian of the transform is 1, which implies no change in volume (incompressibility [Rohlfing T, Maurer C R, Bluemke D A, Jacobs M A (2003) IEEE Trans Med Imaging 22:730-741]). The final rigidity penalty is a weighted combination of the above conditions for e.g., in the ratio 100:1:10. The region where this needs to be imposed, the tumor region in our case, is specified via a 0, 1 tumor segmentation mask. For B-spline s transformations, the rigidity penalty is imposed via the control points, taking advantage of the local support of B-splines. E.g., to ensure rigidity at a point x, all the control points in the local support of the B-spline that influence x are forced to be rigid. Rigidity of the control points ensures rigidity of the object.
Overall registration optimization cost function: The overall registration optimization cost function included the following terms: similarity metric, the bending energy regularization, the rigidity penalty, and the ROI (tumor/ablation) centroid matching term for rigid registration, all of whose weights were set to 1.

All registrations were performed using the SimpleElastix image registration library [Klein S, et al (2009) IEEE Trans Med Imaging 29:196-205; Marstal K, Berendsen F, Staring M, Klein S (2016) SimpleElastix: A user-friendly, multi-lingual library for medical image registration. In: Proceedings of the IEEE conference on computer vision and pattern recognition workshops. pp 134-142]. We set several of the registration parameters above following Klein S, et al (2009) IEEE Trans Med Imaging 29:196-205.

### Example 2: Efficacy of the Methods of the Present Technology in Predicting Pre-Operative Lung Ablation

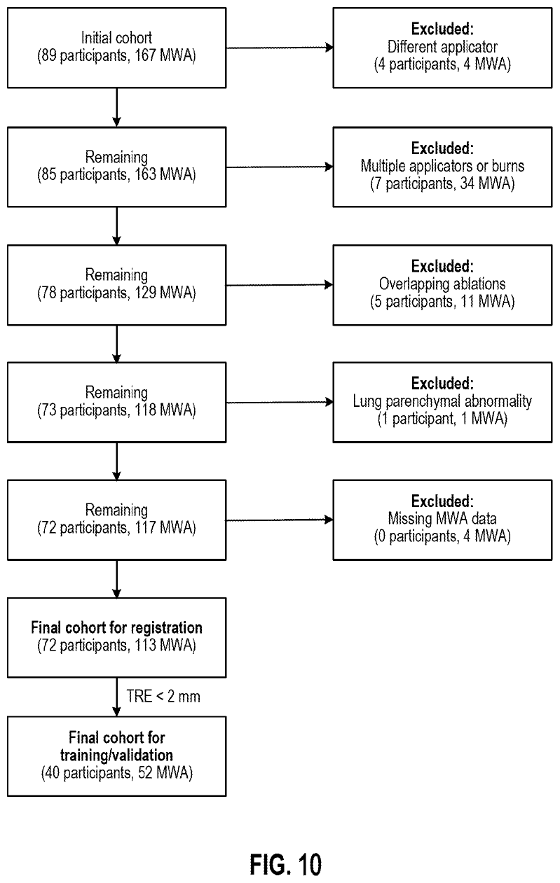

After exclusions, there were 72 participants (median age 57 years, interquartile range [IQR] 47-69; 41 women) with 113 MWA procedures (FIG. 10). MWAs with TRE less than 2 mm were used for training and validation of the algorithm, resulting in 52 unique ablations (Table 1). The median ablation power was 65 watts (range 20-65 watts), and the median duration was five minutes (range 1-10 minutes). There were 17 MWA performed at 65 Watts and five minutes. Ablation zone volume and shape variability are summarized in Table 2 and FIG. 11A. Vendor volume, SVR, and sphericity were significantly different from the median sample volume, SVR, and sphericity, respectively (p<0.05, p<0.01, p<0.001).

TABLE 2
Difference between observed ablation zones and vendor
model shape characteristics and volume. The observed
values were not found to be normally distributed.

| | Vendor | Observed | | |
|---|---|---|---|---|
| Characteristic | model | mean | Variance | p-value |
| Sphericity | 0.93 | 0.67 | 0.002 | <0.001 |
| Elongation | 0.61 | 0.68 | 0.031 | 0.11 |
| Surface-to-volume | 0.31 | 0.41 | 0.02 | <0.008 |
| Volume | 4.24 | 8.91 | 42.3 | <0.015 |

### Bias and Variability of Volume and Shape for Predicted and Vendor Models

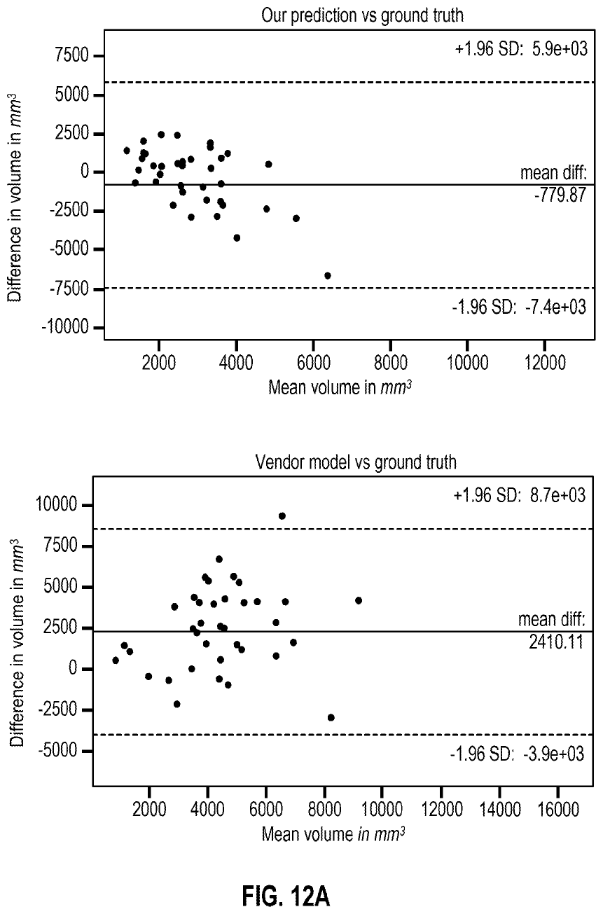

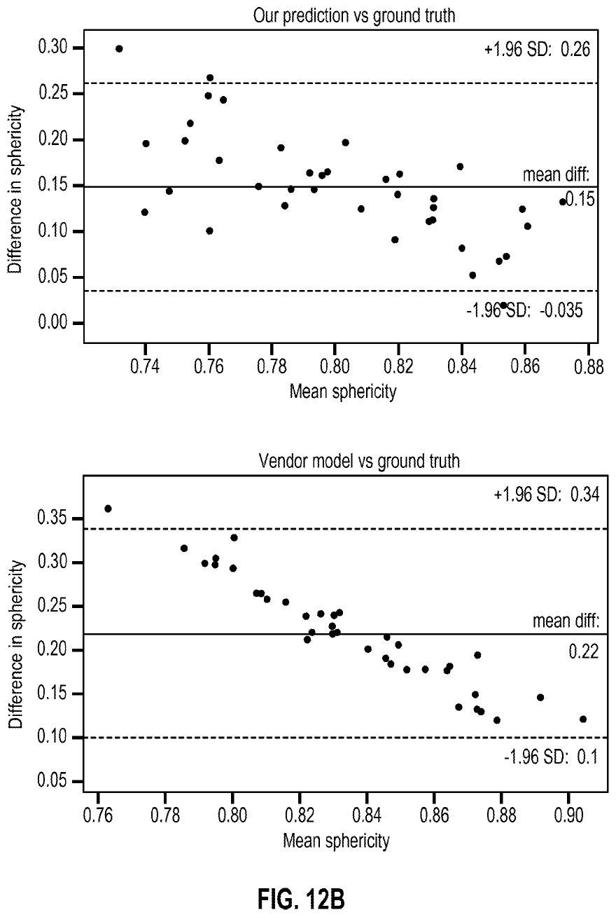

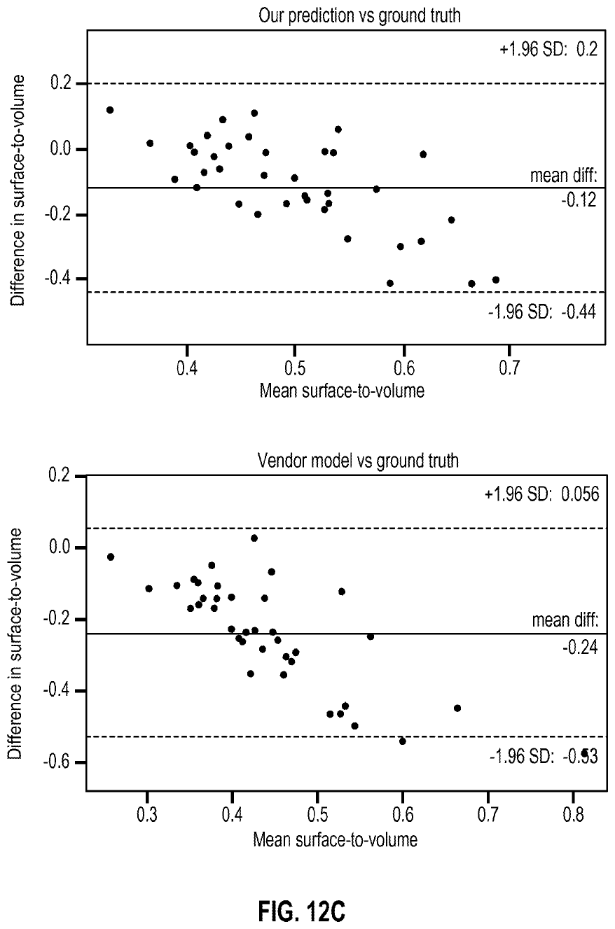

Bland-Altman plots comparing the volumes and shape characteristics of our prediction and vendor model against the true ablation zone are shown in FIG. 12. The volume bias (FIG. 12A) of our prediction (−780) was lower compared to the vendor model (2410) and this difference was significant (p<0.001). Assuming differences from ground truth to be normally distributed (y-axis), the bias of our prediction was not different from the line of equality with ground truth (0 bias) (p=0.169), whereas the vendor model bias was significant (p<0.001). The range in the LOA of our model was smaller than the vendor model and this difference was significant (p<0.001). The sphericity (FIG. 12B) and surface-to-volume ratio (FIG. 12C) biases of our prediction were lower compared to the vendor model (0.15 vs 0.22 and −0.12 vs −0.24, p<0.001). Volume and shape differences demonstrate the better agreement of our prediction with the true ablation zone compared to that of the vendor.

### Importance of Performing Registration Step on Each Sample for Training the Algorithm

The avg Dice score of our method on well registered data (lower target registration error of <2 mm) is 0.6. Whereas on poorly registered data (higher target registration error of <12 mm), the avg Dice score is 0.54, which is the same as that of the applicator vendor prediction, the current clinical standard. Hence registration is a useful step for ablation prediction.

### Dice Score, Precision, Recall

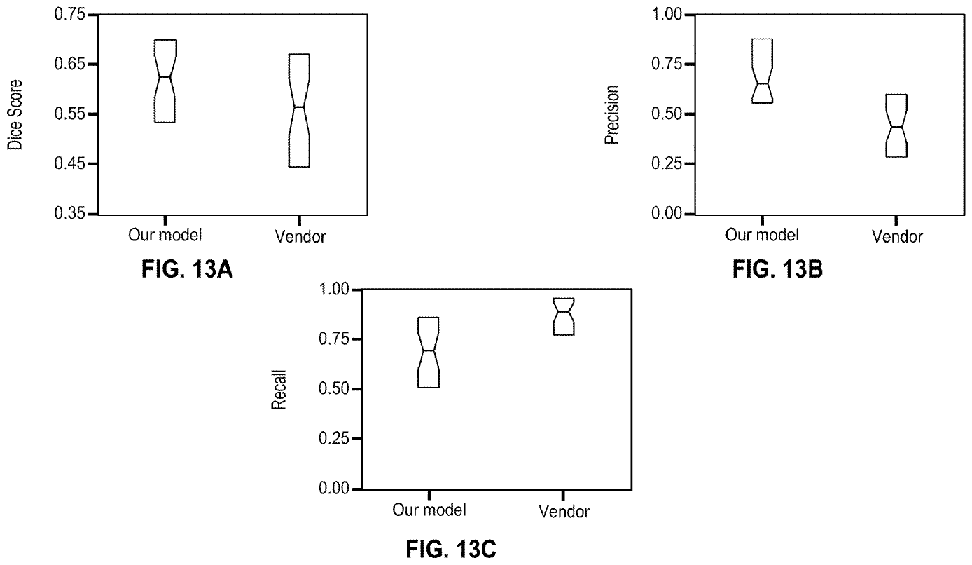

We calculated Dice scores of our prediction and the vendor model with the true ablation zones (FIG. 13A, Table 3). The median and mean Dice scores on the test set were 0.62 and 0.60±0.12 [CI: 0.56-0.64] for our model compared with 0.56 and 0.54±0.16 [CI: 0.49-0.59] for the vendor model, demonstrating an 11% improvement over the vendor model. The vendor model also showed a broader IQR consistent with the large LOA range. We saw improved precision of our model with median and mean scores of 0.65 and 0.67±0.22 [CI: 0.60-0.75] compared with 0.43 and 0.46±0.23 [CI: 0.39-0.54] for the vendor model (FIGS. 13B-13C, and Table 3). The vendor model demonstrated better recall with median and mean scores of 0.89 and 0.84±0.14 [CI: 0.79-0.89] compared with 0.70 and 0.66±0.22 [CI: 0.60-0.73] for our model.

TABLE 3
Dice scores, precision and recall for our model and the vendor model.
In each cell, the top row shows the mean and standard deviation,
whereas the bottom row shows the 95% confidence interval. The method
with the higher score in each column is shown in boldface.

| Method | Dice | Precision | Recall |
|---|---|---|---|
| Ours | 0.60 ± 0.12 | 0.67 ± 0.22 | 0.66 ± 0.22 |
| | [0.56-0.64] | [0.60-0.75] | [0.60-0.73] |
| Vendor | 0.54 ± 0.16 | 0.46 ± 0.23 | 0.84 ± 0.14 |
| | [0.49-0.59] | [0.39-0.54] | [0.79-0.89] |

### Effect of Patient-Specific Local Anatomy

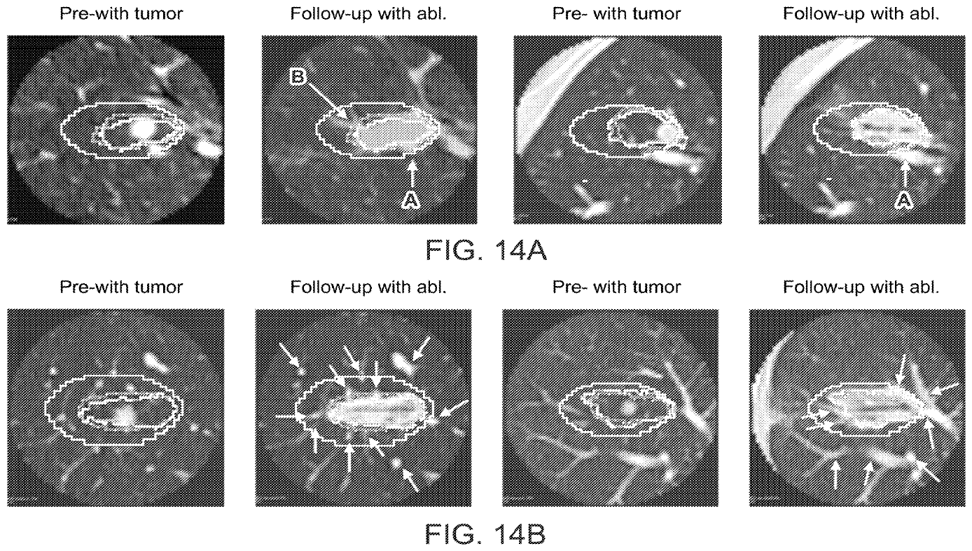

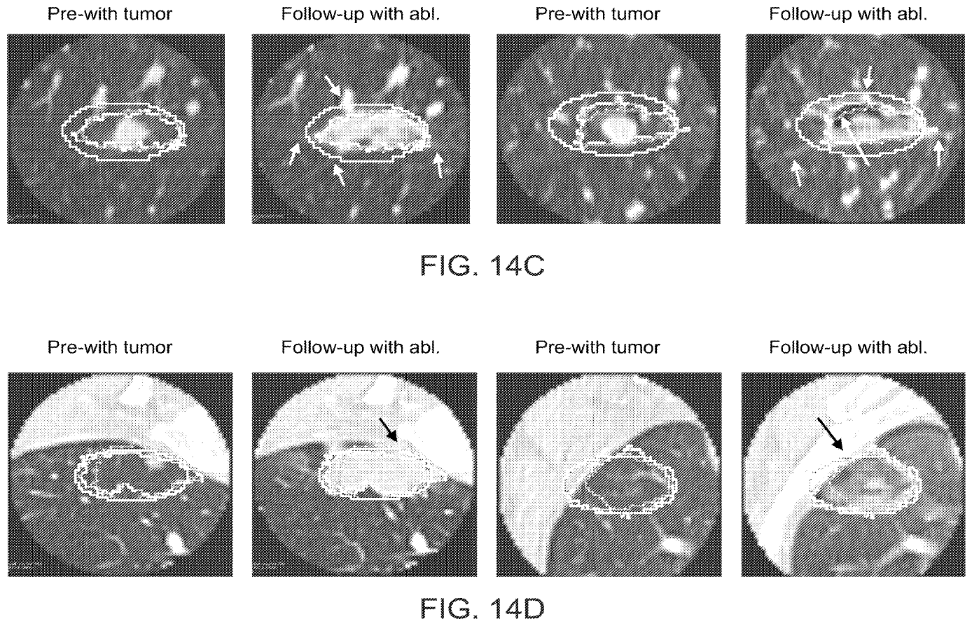

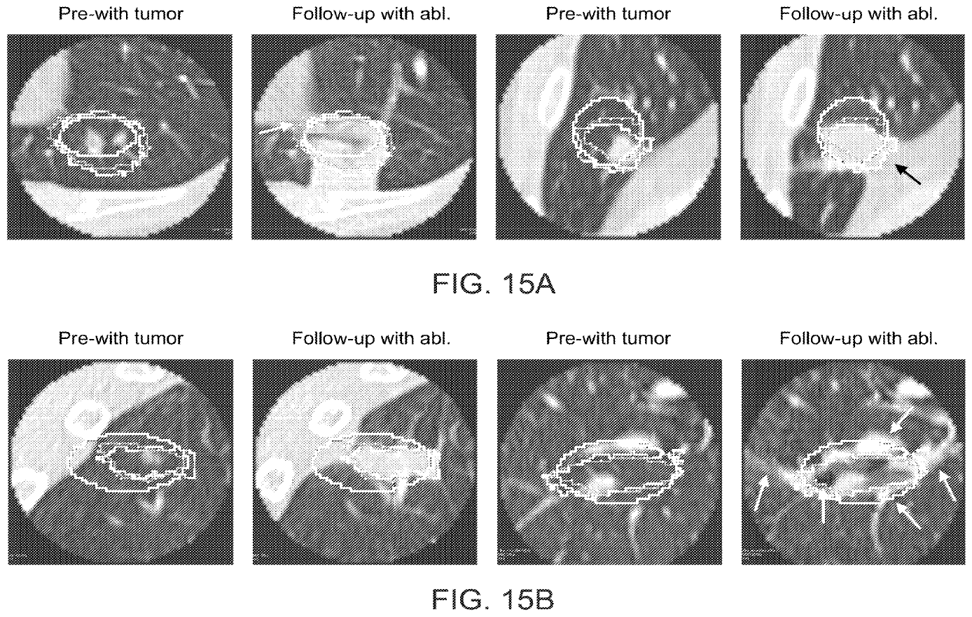

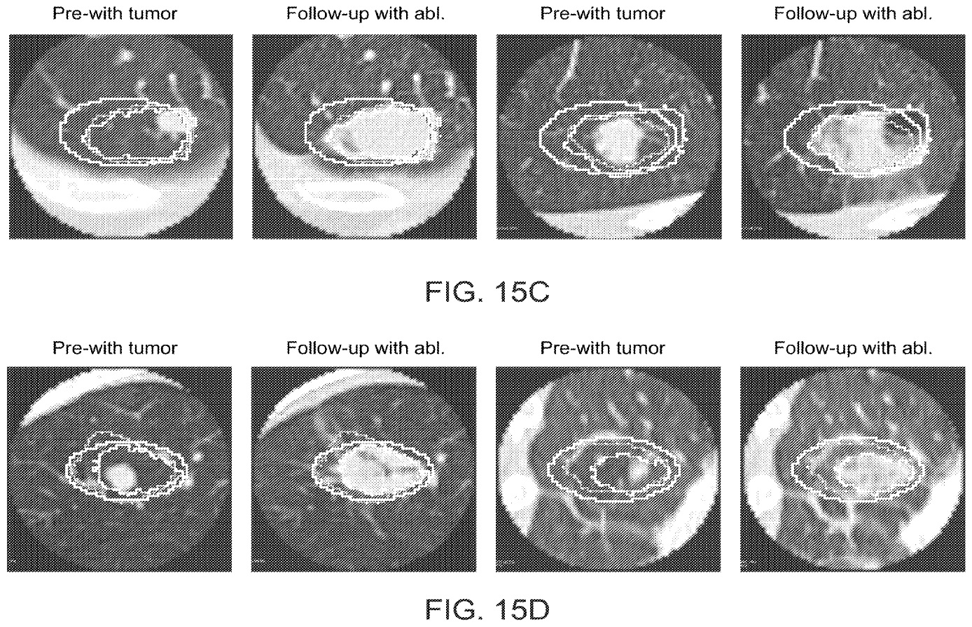

To better understand the quantitative improvement in Dice score, we evaluated the predicted and vendor models in different local anatomical scenarios (FIGS. 14-15). In each example, we show the pre-procedure scan registered to the follow-up post scan (left), the follow-up post scan (right), and mark the ground truth (inner dashed line), predicted model (inner dotted line), and vendor model (outer solid line).

Heat sink effects: FIG. 14A demonstrates two examples of local heat sink effect from adjacent vessels and airways that locally modulate the shape of the ablation zone. Our prediction does not extend into the vessels or airway, similar to the ground truth. In contrast, the vendor model overestimates the ablation zone and extends into the vessels, as well as the background lung parenchyma. FIG. 14B demonstrates two examples of global heat sink effect on ablation zone size and shape. In the left example, multiple small and medium-sized vessels (blue arrows) are seen in the cross section coming in and out of the plane with a cumulative effect of decreasing the overall ablation zone size. The predicted model closely follows the true ablation zone. Notably, the effects of the vessels are not distributed symmetrically around the needle. The vendor model is close to the true ablation zone at the tip of the applicator but overestimates at the back and sides of the applicator. In the right example, multiple vessels (blue arrows) are now seen in plane. Again, the prediction conforms to the asymmetric narrowing and decreased size of the ablation zone.

Borders: FIG. 14C shows two examples demonstrating the effects of fissures, or faint radio-dense lines demarcated by blue arrows on post-procedure images. Notably, fissures are visible on the post-procedure scan but difficult to identify on the pre-procedure scans. The predicted model follows the fissure, whereas the vendor model extends beyond the fissure. FIG. 14D shows two examples of chest wall boundaries, which the predicted model adheres to closely. In the right example, the vendor model extends beyond the boundary and overestimates the ablation zone. FIG. 15A shows examples of mediastinal borders where the predicted model adheres to the local border, whereas the vendor underestimates (left) or overestimates (right) the ablation zone.

Ablation shape: The overall shape of the ablation zone is affected by lung anatomy. We identified different forms of narrowing of the ablation zone cavity relative to the vendor model, and which our predicted model was able to identify. FIG. 15B (left) shows asymmetric narrowing of the ablation zones to which our model adheres closely, but that the vendor model overestimates. In the right example, the ablation zone appears rotated relative to the vendor model, possibly related to the adjacent vascular structures, and which our model is able to predict. FIG. 15C (left) shows our prediction narrowing only at the rear end of the ablation, similar to ground truth.

Areas for future improvements: FIG. 15C (right) shows a case in which the ablation zone contains a large cavity that our model excludes. A similar cavity also excluded by our model is seen in FIG. 15C (arrow). FIG. 15D (left) shows an example in which our prediction extends too far and wide along the back of the probe. Close review of the intra-procedure ablation images revealed that the lung was torqued and compressed intraoperatively during applicator positioning. FIG. 15D (right) shows an example of confounding background anatomy wherein our model adheres to scars within the lung parenchyma at the back of the ablation that have an ablation border-like appearance. However, these specific scenarios are potentially surmountable with sufficient relevant training data.

### Advantages of the Present Model Over the Existing Biophysical Models

Our method directly incorporates patient specific observed data via pre/intra/post CT scans into ablation prediction, whereas the current biophysical models depend on a multitude of tissue properties such as electrical, thermal, and physiological of the patient that are impractical to measure on a case-by-case or location-by-location basis. The best example of tissue properties applied in biophysical models was reported by Sebek et al.³⁰ The authors incorporated tissue dielectric properties into their biophysical model and showed some results on in vivo porcine lung. This approach only accounts for differences between lung and tumor. However, there are many complex boundaries in the surrounding lung including three different types of vessels (pulmonary artery, pulmonary vein, bronchial artery) with different sizes and complex geometry, airways with different sizes and complex geometry, pleural surfaces (including lobar boundaries, chest wall boundaries, and mediastinal boundaries) and segmental surfaces with complex geometry. There is no known methodology to account for all of these parameters and their complex geometries in a biophysical model. Moreover, the tissue properties required for accurate biophysical modeling vary across patients, organs and it is impossible to measure them at every location inside the organ. For every parameter added to the biophysical model, the computational complexity increases exponentially and therefore makes this approach unfeasible for clinical use. Notably, Sebek et al conceded “the experimental ablation zone that extended beyond the simulation bounds may be attributed to heterogeneity within the lung parenchyma due to discrete structures such as airway walls.” Thus, existing biophysical models are problematic because even a small difference of several mm may mean the difference between local recurrence or cure. See Gao S, Stein S, Petre E N, et al. Cardiovascular and interventional radiology. 2018; 41(2):253-259.

Additionally, our method directly optimizes the likelihood of the observed output ablation zones via backpropagation algorithm, whereas the current biophysical models lack such feedback. Our method is faster than bio physical modeling (inference takes fraction of a second vs hours for detailed bio-physical simulation), making the present model suitable for real-time clinical implementation. Further, our method is directly validated on the observed in vivo clinical ablation zones whereas bio-physical models lack in vivo clinical validation. Prior biophysical modeling studies generally make oversimplified assumptions such as homogeneous tissue properties (vs heterogeneous), 2D anatomical geometries (vs 3D), simple applicator configurations, and have only validated on ex vivo (vs in vivo) tissue.

Our method directly models the ablation zone on the follow-up CT scan 1-3 month post ablation, which is the standard time point for treatment verification clinically. The first follow-up CT (typically performed at 1 month post ablation) is the established baseline for evaluating the ablation zone after lung ablation. This is the scan that is used to determine the minimum margin, which has been associated with local recurrence after lung ablation (Gao S, Stein S, Petre E N, et al. Cardiovascular and interventional radiology. 2018; 41(2):253-259; Yan P et al., BMC Medical Imaging. 2021; 21(1):96). Notably, we have previously reported and quantified the extent of tissue contraction observed on these one month scans which are the gold standard for margin assessment (Dev A, Keshavamurthy K N, Salkin R, et al. Abstract No. 124 Journal of Vascular & Interventional Radiology. 2022; 33(6):S58-S59). Hence, at the follow-up stage, the ablation zone is partially influenced by other biological/chemical processes as well (e.g., involution due to healing), in addition to the necrosis induced by microwave heating. In contrast, biophysical models predominantly only account for thermal dose and the ensuing tissue cell death, and do not explicitly model these additional biological/chemical factors at the follow-up stage, thus reducing their predicting accuracy.

Moreover, our approach is not restricted to the class of functions that can be modeled by the coupled partial differential equations of the biophysical models. Indeed, the complex shape metrics for tumors impacts accuracy of ablation plan. We have previously reported on the variability and complexity of ablation zone shapes (Dev A, Keshavamurthy K N, Solomon S B, Ziv E. Quantitative analysis of microwave lung ablation zones. Paper presented at: Society of Interventional Oncology 2021; San Francisco, CA; Salkin R, Keshava Murthy K N, Dev A, et al. Journal of Vascular and Interventional Radiology. 2022; 33(6 Suppl). However, the vendor model is a simple ellipsoid and the biophysical model will tend to smooth the boundaries. Neither of these models can appropriately model the complex shapes of true ablation zones, which include sharp edges, discontinuities, and concavities that are a direct result of the local anatomy. Our method is thus capable of accounting for complicated intra-procedure effects observed in real ablations such as atelectasis, pneumothorax, tissue torquing etc. and varying anatomical geometry such as organ/lobar boundaries, that are visible on the scans and that can cause sharp ablation boundaries.

### CONCLUSIONS

We have presented a deep learning model to predict lung ablation zones as they appear on follow-up scan based on pre-procedure imaging and ablation parameters. Compared with the vendor model, we demonstrated higher Dice scores, decreased volume variability, and decreased bias in volume and shape. Our model is also able to predict the ablation zone in the context of challenging scenarios, including heat sink effects, boundaries not visible on input images, and variations in ablation zone shape.

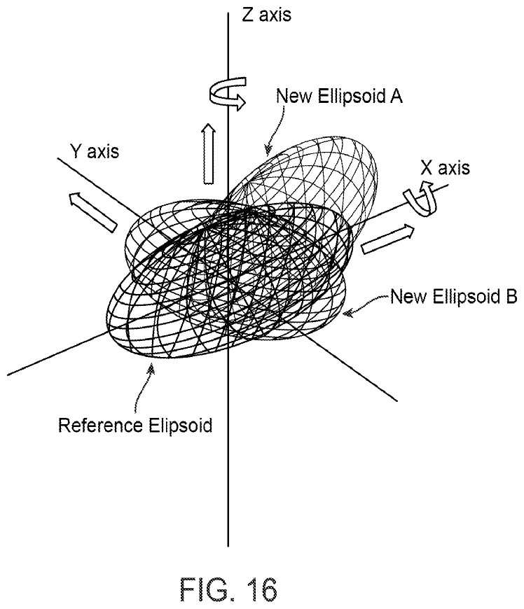

Moreover, we note that the improvement in Dice performance is likely an underestimate. The added step of registration for our model introduces a bias against our model relative to the vendor model. To quantify the effect of mis-registration on Dice score, we computed Dice scores between ellipsoids of similar dimensions but at different relative positions and orientations. The ellipsoid dimensions were chosen such that they corresponded with the vendor ablation zone at 65 W and 5 min, as these were the median ablation power and duration settings in our data set. FIG. 16 illustrates the procedure. The reference ellipsoid was placed at the origin, with the major axis oriented along the x axis. New ellipsoids were then created by perturbing the reference ellipsoid via translations along different directions as well as rotations about different axis. All rotations were first applied at the origin followed by translations to different locations. The translations varied from 0-5 mm and the rotations varied between 0°-45°. Both in-plane and out-of-plane transformations were performed. Dice scores were then computed in each of these scenarios between the perturbed and the reference ellipsoid as shown in Table 4.

TABLE 4
Impact of misregistration on Dice score. Listed are Dice
scores between ellipsoids that are misregistered by
0-5 mm in position and by 0 to 45° in orientation.
In-plane rotations and translations

| | | | | 2 mm |
|---|---|---|---|---|
| Angle of | | 2 mm | 2 mm | translation |
| rotation | No | translation | translation | along x = y |
| about z axis | translation | along x axis | along y axis | line in-plane |
| 0° | 1.0 | 0.91 | 0.84 | 0.87 |
| 15° | 0.88 | 0.87 | 0.83 | 0.87 |
| 45° | 0.71 | 0.70 | 0.70 | 0.70 |
| | | | | 3 mm |
| Angle of | | 3 mm | 3 mm | translation |
| rotation | No | translation | translation | along x = y |
| about z axis | translation | along x axis | along y axis | line in-plane |
| 0° | 1.0 | 0.87 | 0.76 | 0.81 |
| 15° | 0.88 | 0.84 | 0.76 | 0.85 |
| 45° | 0.71 | 0.69 | 0.68 | 0.69 |
| | | | | 5 mm |
| Angle of | | 5 mm | 5 mm | translation |
| rotation | No | translation | translation | along x = y |
| about z axis | translation | along x axis | along y axis | line in-plane |
| 0° | 1.0 | 0.79 | 0.61 | 0.69 |
| 15° | 0.88 | 0.77 | 0.62 | 0.77 |
| 45° | 0.71 | 0.65 | 0.62 | 0.65 |

Out-of-plane rotations and translation

| Angle of | | | | |
|---|---|---|---|---|
| rotation | | 2 mm | 3 mm | 5 mm |
| about z axis | | translation | translation | translation |
| and angle of | | along | along | along |
| rotation from | | oriented | oriented | oriented |
| x-y plane in | No | direction | direction | direction |
| series | translation | in 3D | in 3D | in 3D |
| 15° | 0.84 | 0.83 | 0.81 | 0.75 |
| 45° | 0.65 | 0.64 | 0.64 | 0.60 |

We saw large effects on the Dice score (10-20% decrease) even with small perturbations in translation (2 mm) or angle of rotation) (15°). Notably, unlike our models, the vendor model is applied directly to the follow-up post scan (from which the applicator coordinates are extracted) and does not involve any registration and is therefore not subject to this registration effect. Thus, the true improvement over the vendor model in the Dice score is likely higher.

Clinically, the trade-off between precision and recall is significant. A bias to overestimate the ablation zone increases recall. But this means the operator will undertreat the tumor, resulting in higher local recurrence rates. Conversely, a bias to underestimate the ablation zone can increase precision, but may result in overly aggressive treatment and potential complications. It is therefore important to establish a balance between precision and recall. The vendor model shows a striking mismatch between precision and recall. The low precision and high recall of the vendor model translates clinically to overestimation of the ablation zone, incomplete treatment, and high local recurrence. This was also demonstrated in the significant positive bias of the vendor model in the Bland-Altman plot. Our model showed no bias, as well as marked improvement in precision at the cost of some decrease in recall, thereby averting both tumor undertreatment and local recurrence. Depending on location and proximity to critical structures, one could tune the threshold to modulate the precision/recall trade-off.

Our approach offers several advantages over first principle physical models. We directly incorporate patient-specific observed data via CT scans, whereas biophysical models depend on multiple tissue properties that are impractical to measure.^{11,30} We directly optimize the likelihood of the observed ablation zones via backpropagation,²⁹ whereas biophysical models lack such feedback. Our algorithm is faster (inference takes fractions of a second),¹¹ making it suitable for clinical implementation. We directly model the ablation zone on the follow-up CT, the standard time point for treatment verification, therefore incorporating post-ablation processes, such as tissue contraction and healing.^{31,32} Biophysical models only account for thermal dose and ensuing tissue cell death, and do not explicitly model additional factors at follow-up. Our approach is not restricted to the class of functions that can be modeled by the coupled partial differential equations of the physical models.

Our method has several important clinical applications.^{4,33} Pre-procedure planning with our model can be used to establish applicator trajectory, as well as power and duration to optimize the margin and decrease local recurrence. The model can be used to identify “weak points” in the ablation that may require additional treatment. The rapidity with which the inference can be performed allows for real-time clinical tuning by the operator. Intraoperative feedback regarding the ablation zone margin has been used to improve outcomes in liver ablations, but requires additional biopsies, thereby increasing the risk of the procedure.³⁴ Using our method, these options would also be cost-effective by decreasing incomplete ablations and local recurrences and therefore avoiding repeat procedures. By pre-defining a minimum ablation margin around the nodule, we also envision a scenario wherein a set of “optimal solutions” can be posed by the model and from which the operator can select. Finally, our approach, can be applied more broadly to other organs and other treatment modalities.

We have presented a patient-specific deep learning model to predict the lung ablation zone with the purpose of decreasing local recurrence and improving outcomes post-MWA. The work demonstrates a novel application of deep learning for a common yet unresolved clinical problem. It yields improved results over the current standards and, to our knowledge, the best results to date.

### Example 3: Newer Improved Hierarchical Registration Method for Ablation Workflows

The pre and post ablation procedure CT scans have the tumor and ablation zones respectively. Here, by pre procedure scan, we refer to patient imaging scans performed immediately prior to several weeks or months prior to the ablation procedure. Similarly, by post procedure scan, we refer to the patient imaging scan performed immediately post to several or months post procedure. The anatomy in the two scans would not be matched since the patient may be in a different position, different phases of inspiration, or may have moved between the two scans. Image registration matches the anatomy in the two scans, establishing a one-to-one correspondence between the voxels (volumetric pixels) in the two scans. This is a step that enables 1) comparison of tumor and ablated region and measure treatment success, i.e., if the tumor plus a pre-determined neighboring margin around tumor was burned completely by the ablation (completely covered by the ablation zone); 2) learning the predictive relationship between the tumor and ablated region in the pre and post procedure scans respectively. This allows to train an artificial intelligence algorithm to predict the ablation zone (as would be seen on the post procedure scan) from pre-procedure scan and ablation treatment parameters.

An improved image registration procedure for ablation workflows was developed and the procedure obtained better accuracy compared to the method disclosed in Examples 1-2:

Let the post ablation procedure scan be the fixed image and the pre ablation procedure scan be the moving image. The goal is to register or match the moving image to the fixed image, i.e., the pre to the post procedure scan. The new registration method is based on a multi-stage hierarchical registration process. The key idea is to start with a coarse registration and perform finer and more focused registrations in subsequent stages.

In the first stage, we perform an affine registration between the pre and post procedure scans to obtain a coarse global initial alignment of the anatomy. Zitova B, Flusser J. Image Vis Comput. 2003; 21(11):977-1000. This accounts for the differences in inspiration between the scans and aligns the anatomy globally. This step provides a good initialization for the subsequent finer deformable registrations.

In the second stage, we perform a multi-resolution deformable image registration to account for the non-rigid motion of the lung anatomy between the pre and post procedure scans. Klein S et al., IEEE Trans Med Imaging. 2009; 29(1):196-205; Lester H, Pattern Recognit. 1999; 32(1):129-149. This also performs a finer match between the anatomies while allowing for deformable changes due to patient motion, breathing etc.

In the third stage, we perform another multi-resolution deformable image registration between the scans, where instead of matching the entire scans as in the previous step, we focus the registration to match the regions inside the lung of interest, i.e., the lung where the ablation was performed. Staring M et al., Medical Image Analysis for the Clinic: A Grand Challenge. 2010:73-79. This local registration is performed using a mask that covers only the lung parenchyma on the post procedure scan, i.e., the fixed image. This step aids in obtaining a finer match between the local anatomy in the region of interest such as between blood vessels surrounding the ablation zone, and avoids the registration being unnecessarily influenced by other far away anatomy that is unrelated to the ablated region such as the non-participating lung (other lung), spine, ribs etc.

In the fourth stage, we focus the registration even more by performing a multi-resolution deformable image registration between only the closest neighborhood around the ablation zone. This can be done in multiple ways: a) we can focus the registration on the specific lobe of the lung that has the ablation zone by using a lung lobe mask; b) we can focus the registration on the region immediately surrounding the ablation zone using a mask of the same shape as the ablation zone with certain radial thickness surrounding it. In this work, we only performed b), i.e., we focused the registration on the immediate neighborhood of the ablation zone. But we would like to note that both steps a) and b) can be performed in series, which potentially can result in higher accuracy than using only one of them.

In the fifth stage, we can further focus the registration by ensuring that it matches known anatomical points/landmarks in the two scans in addition to matching image intensities. These can include blood vessel bifurcation points, existing calcifications etc. These points can either be picked automatically with some manual QA or fully manually and used for matching. This step would further constrain the registration by ensuring match between known anatomical locations.

We note that while the above multi-stage methodology presents a generic hierarchical registration procedure for ablation workflows, the order of some of the steps, particularly the last one can be changed. For example, the fifth stage of landmark based registration can be performed earlier too.

We also note that an important pre-processing step for all the above registration stages is loss function masking. This essentially removes the ablated region (ablation zone) in the post procedure scan from participating in the registration procedure by masking out such regions in post procedure scans and discounting them from the registration optimization cost function. More generally, we perform loss function masking for any regions in the post procedure scans that is different from pre-procedure scans. This is to ensure such regions, for e.g., the ablation zone in post procedure scans, does not get warped to match to the tumor in the pre-procedure scan which is undesired. Only anatomy outside of such regions are subject to the registration procedure and matched between pre and post scans.

Below we describe the various parameters of the registration for the above procedure. We follow the methods described in Klein S et al., IEEE Trans Med Imaging. 2009; 29(1):196-205; Staring M et al., Medical Image Analysis for the Clinic: A Grand Challenge. 2010:73-79. For setting several of the registration parameters. While we have made certain parametric choices, they are generalizable with other similar parameters, value settings or even different combinations of the below listed method attributes.

### Parameters Used for Registration:

Masking: The ablation zone in the post procedure scan is removed from participating in the registration process by masking it out from the image and the registration loss functions. More generally, we perform loss function masking for any regions in the post procedure scans that is different from pre-procedure scans. This is done by first segmenting out such regions and then inverting the segmentation to obtain a mask that excludes such regions. The lung and lung lobe masks for the 3^{rd} and 4^{th} stages are extracted using semi-automatic approaches but can be fully automated as well. These masks are also dilated by a small radius to make sure the lung and fissure boundaries are included in the mask, so that the boundaries can be matched too. The neighboring anatomical mask surrounding the ablation zone for the 4^{th} stage was obtained by dilating the ablation zone mask by a certain radius.

Image similarity metric: image mutual information or normalized cross correlation can be used as the image similarity metric to compute similarity between the moving and fixed images (Klein S et al. IEEE Trans Med Imaging. 2009; 29(1):196-205; Viola P, Wells III W M. Int J Comput Vis. 1997; 24(2):137-154); for mutual information, the number of intensity histogram bins for computing the joint distribution was chosen to be 32.

Optimizer: stochastic gradient descent, where “step size” was adapted as a function of the similarity between the gradient directions in the current and the previous iterations (Klein S et al. Int J Comput Vis. 2009; 81:227-239). The maximum number of iterations was set to 256.

Image sampler: image intensities were sampled from 2048 locations (including sub-voxel) from the image regions inside the specified registration masks, with new spatial samples selected in every iteration.

Image interpolation: We used tri-linear interpolation during the registration. For computing the final transformed image, B-spline based cubic interpolation is used for better quality.

Non-rigid/deformable registration: We utilize the free form deformation model (FFD) based on B-splines (Rueckert D et al., IEEE Trans Med Imaging. 1999; 18(8):712-721) as the transformation function. FFDs are defined by a grid of control points which can be used to manipulate the shape of 3D objects. Using cubic B-splines, a hyperpatch is defined by the control points in 3D that is smooth and continuous. Moving the control points of the FFD deforms the B-spline transformation function, which in turn results in smooth deformations of the underlying fixed image pixel grid. The parameters of the transform are the positions of the control points, which are optimized during the registration to maximize the similarity between the deformed moving and fixed images. The registration is performed in a multi-grid setting with the grid size adapted from low to high resolution, i.e., coarse to fine, such that finer structures are matched in higher resolutions. For matching landmark points in combination with matching image intensities in stage 5, the thin plate spline transformation function can be utilized to perform the non-rigid registration. Qiu Z et al., In: 2009 WRI World Congress on Computer Science and Information Engineering. Vol 2; 2009:522-527.

Regularization: We utilized the bending energy as our regularizer (Rueckert D et al., IEEE Trans Med Imaging. 1999; 18(8):712-721; Wahba G. Spline Models for Observational Data. SIAM; 1990), which penalizes abrupt variations in the transformation (e.g., high expansion followed by high compression) and can avoid folding.

Multi-resolution strategies: A coarse-to-fine hierarchical image registration at multiple resolutions was performed, where images were smoothed using specified Gaussian kernels to different resolutions and registered incrementally starting with the lowest. Lester H, Arridge SR. Pattern Recognit. 1999; 32(1):129-149.

This can also be implemented with Gaussian pyramids and subsampling. Staring M et al., Medical Image Analysis for the Clinic: A Grand Challenge. Published online 2010:73-79. The lowest resolutions reduce the number of local optima, increasing the likelihood of finding the global optima. This strategy aligns large structures first, with the finer details introduced and matched at successively higher resolutions. The number of resolutions was chosen to be 5, with the registration proceeding from the lower to the higher resolutions in succession. The lower resolution images were obtained by smoothing the image with a Gaussian of σ=0.5 in the finest resolution and increased by a factor of 2 in each successive lower resolution. A similar strategy is followed for the B-spline transformation, where the complexity of the transform (i.e., the number of individual parameters in the transform) is increased from low to high during successive registrations at increasingly higher resolution levels. Klein S et al., IEEE Trans Med Imaging. 2009; 29(1):196-205; Rueckert D et al., IEEE Trans Med Imaging. 1999; 18(8):712-721. At lower image resolutions, a coarse B-spline control point grid is used with higher spacing between control points; this allows for only coarse deformations, aiding to match larger global structures. Whereas at higher resolutions, a finer grid is used with lesser spacing between the control points; this allows for local deformations, aiding to match smaller local structures in the images.

Rigidity penalty on tumor region: While non-rigid registration is to match the anatomy surrounding the tumor in the pre and ablation zone in the follow up post scans, we desire the tumor area itself to not undergo large deformations. Preserving volume and shape morphology of the tumor can be done to learn a meaningful relationship with the ablation zone. We achieve this by imposing a rigidity penalty on the tumor region in the pre scan following Staring et al., Med Phys. 2007; 34(11):4098-4108.

The specific constraints may include: 1) Affinity condition: This condition ensures that the second order partial derivatives of the transform with respect to the spatial coordinates is 0, as required by a rigid transform (and more generally an affine transform); 2) Orthonormality condition: This condition enforces the property that the rotation matrix should be orthonormal; and 3) Properness: This condition ensures that the determinant of the rotation matrix is 1. This is equivalent to imposing the determinant of the Jacobian of the transform is 1, which implies no change in volume (incompressibility; Rohlfing T et al., IEEE Trans Med Imaging. 2003; 22(6):730-741). The final rigidity penalty is a weighted combination of the above conditions, e.g., in the ratio 100:1:10. The region where this needs to be imposed, the tumor region in our case, is specified via a 0, 1 tumor segmentation mask. For B-splines transformations, the rigidity penalty is imposed via the control points, taking advantage of the local support of B-splines. For example, to ensure rigidity at a point x, all the control points in the local support of the B-spline that influence x are forced to be rigid. Rigidity of the control points ensures rigidity of the object.

Overall registration optimization cost function: The overall registration optimization cost function included the following terms: similarity metric, the bending energy regularization, the rigidity penalty, all of whose weights were set to 1. But this can be optimized further. All registrations were performed using the SimpleElastix image registration library. Klein S, et al., IEEE Trans Med Imaging. 2009; 29(1):196-205; Marstal K et al., In: Proceedings of the IEEE Conference on Computer Vision and Pattern Recognition Workshops. 2016:134-142.

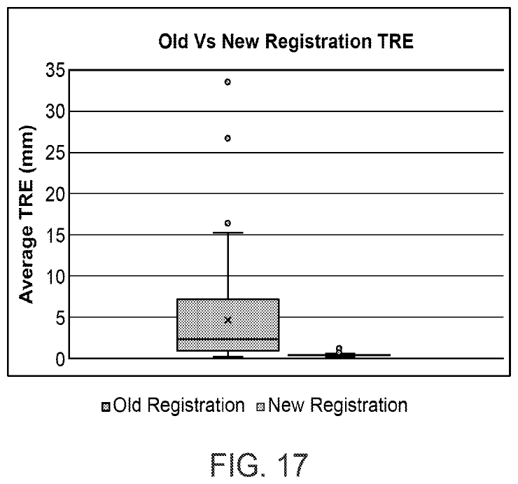

FIG. 17 shows the results of the older versus the newer registration method. Note that in the newer registration method shown below, we only performed the first 3 stages of the above described multi-stage procedure and we already see much higher improvement in TRE scores compared to the older method (median old target registration error (TRE)=2.26 mm vs median new TRE=0.297 mm; i.e., ~8 fold improvement in registration accuracy, p<0.01). Note that lower TRE indicates higher registration accuracy. We expect that performing all 5 stages of the above described multi-stage registration procedure would result in even higher registration accuracy.

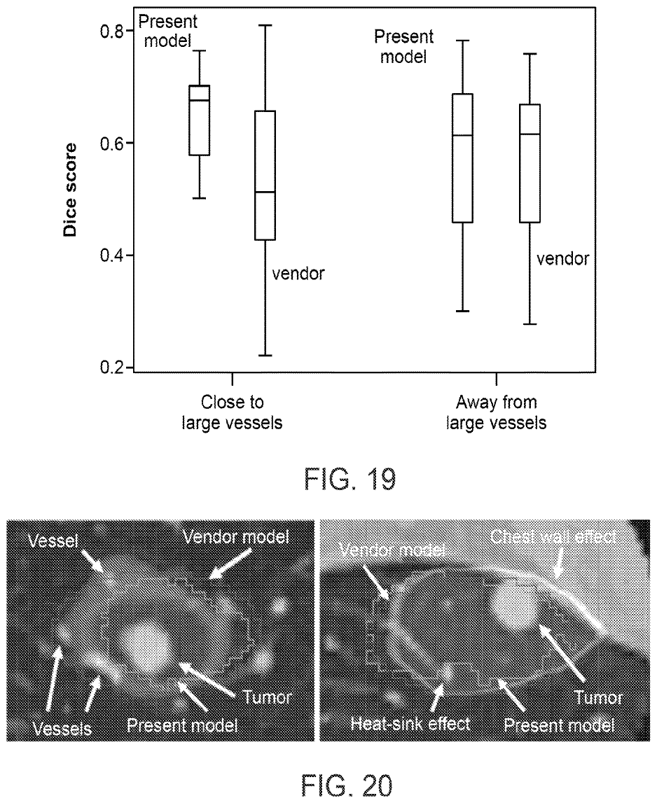

Model accounts for patient-specific factors: The improvement in Dice score is most striking in laparoscopic microwave ablation (LMWA) near large vessels (FIG. 19) demonstrating the superior ability of our method to account for local anatomical effects. This is also demonstrated in challenging anatomical scenarios, supporting the veracity, sensitivity and accuracy of the prediction models disclosed herein.

Model predictions are “explained” by relevant anatomical regions: The black box critique of all CNN based models is that the features that the network uses to make a decision, or an output are hidden. Ronneberger O et al., In: Medical Image Computing and Computer-Assisted Intervention—MICCAI 2015: 18^{th} International Conference, Munich, Germany, Oct. 5-9, 2015, Proceedings, Part III 18; 2015:234-241). We adapted a well described saliency map generation method called GradCam (Selvaraju R R et al. In: Proceedings of the IEEE International Conference on Computer Vision, 2017:618-626) to the task of dense image prediction. The GradCam uses the gradients of the output with respect to the convolutional features to produce a localization map that highlights the regions that the network used to make the prediction. In FIG. 20, we show the output of GradCam applied to our model on test set examples. The pixels around the local anatomy of the tumor contribute the most to the prediction as they are the most relevant regions for ablation. Also, the activation maps extend into vessels and chest walls which inform the ablation boundaries and account for “heat-sink” effects and heterogeneous organ characteristics. These results confirm that our algorithm was influenced by relevant regions in its decision making and not by spurious activations or shortcut learning.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

### REFERENCES

1. Simon C J, Dupuy D E, Mayo-Smith W W. Microwave ablation: principles and applications. Radiographics. 2005; 25(suppl_1):S69-S83.
2. Kwan S W, Mortell K E, Hippe D S, Brunner M C. An economic analysis of sublobar resection versus thermal ablation for early-stage non-small-cell lung cancer. Journal of Vascular and Interventional Radiology. 2014; 25(10):1558-1564.
3. Binkley M S, Shrager J B, Chaudhuri A, et al. Time course and predictive factors for lung volume reduction following stereotactic ablative radiotherapy (SABR) of lung tumors. Radiation Oncology. 2016; 11(1):1-8.
4. Dupuy D E, Fernando H C, Hillman S, et al. Radiofrequency ablation of stage IA non-small cell lung cancer in medically inoperable patients: Results from the American College of Surgeons Oncology Group Z 4033 (Alliance) trial. Cancer. 2015; 121(19):3491-3498.
5. Gao S, Stein S, Petre E N, et al. Micropapillary and/or solid histologic subtype based on pre-treatment biopsy predicts local recurrence after thermal ablation of lung adenocarcinoma. Cardiovasc Intervent Radiol. 2018; 41(2):253-259.
6. Blackmon S H, Sterner R M, Eiken P W, et al. Technical and safety performance of CT-guided percutaneous microwave ablation for lung tumors: an ablate and resect study. J Thorac Dis. 2021; 13(12):6827.
7. Gao S, Stein S, Petre E N, et al. Micropapillary and/or solid histologic subtype based on pre-treatment biopsy predicts local recurrence after thermal ablation of lung adenocarcinoma. Cardiovasc Intervent Radiol. 2018; 41(2):253-259.
8. Dev A, Keshavamurthy K N, Salkin R, et al. Quantitative analysis of tissue contraction and volume variability of lung microwave ablation zones. In: Journal of Vascular & Interventional Radiology; 2022:33(6):S58-S59.
9. Dev A, Keshavamurthy K N, Solomon S B, Ziv E. Quantitative analysis of microwave lung ablation zones. In: Presented at: Society of Interventional Oncology; 2021.
10. Huber T C, Miller G, Patrie J, Angle J F. Relationship of Antenna Work and Ablation Cavity Volume Following Percutaneous Microwave Ablation of Hepatic Tumors. Journal of Vascular and Interventional Radiology. 2021; 32(4):536-543.
11. Prakash P. Theoretical modeling for hepatic microwave ablation. Open Biomed Eng J. 2010; 4:27.
12. Chiang J, Wang P, Brace C L. Computational modelling of microwave tumour ablations. International Journal of Hyperthermia. 2013; 29(4):308-317.
13. Fedorov A, Beichel R, Kalpathy-Cramer J, et al. 3D Slicer as an image computing platform for the Quantitative Imaging Network. Magn Reson Imaging. 2012; 30(9):1323-1341.
14. Rueckert D, Sonoda L I, Hayes C, Hill D L G, Leach M O, Hawkes D J. Nonrigid registration using free-form deformations: application to breast M R images. IEEE Trans Med Imaging. 1999; 18(8):712-721.
15. Lester H, Arridge S R. A survey of hierarchical non-linear medical image registration. Pattern Recognit. 1999; 32(1):129-149.
16. Klein S, Staring M, Murphy K, Viergever M A, Pluim J P W. Elastix: a toolbox for intensity-based medical image registration. IEEE Trans Med Imaging. 2009; 29(1):196-205.
17. Marstal K, Berendsen F, Staring M, Klein S. SimpleElastix: A user-friendly, multi-lingual library for medical image registration. In: Proceedings of the IEEE Conference on Computer Vision and Pattern Recognition Workshops; 2016:134-142.
18. Yaniv Z, Lowekamp B C, Johnson H J, Beare R. SimpleITK image-analysis notebooks: a collaborative environment for education and reproducible research. J Digit Imaging. 2018; 31(3):290-303.
19. Keshava K N, Kimia B B, Cook M, Dupuy D E, Collins S A, Merck D. A methodology to analyze treatment zone geometry and variability of percutaneous thermal ablation. In: Energy-Based Treatment of Tissue and Assessment VIII. Vol 9326. SPIE; 2015:240-247.
20. Isensee F, Jaeger P F, Kohl S A A, Petersen J, Maier-Hein K H. nnU-Net: a self-configuring method for deep learning-based biomedical image segmentation. Nat Methods. 2021; 18 (2): 203-211. Doi: 10.1038/s41592-020-01008-z
21. Ulyanov D, Vedaldi A, Lempitsky V. Instance normalization: The missing ingredient for fast stylization. arXiv preprint arXiv: 160708022. Published online 2016.
22. Maas A L, Hannun A Y, Ng A Y. Rectifier nonlinearities improve neural network acoustic models. In: Proc. Icml. Vol 30. Atlanta, Georgia, USA; 2013:3.
23. Lee C Y, Xie S, Gallagher P, Zhang Z, Tu Z. Deeply-supervised nets. In: Artificial Intelligence and Statistics. PMLR; 2015:562-570.
24. Chen L C, Papandreou G, Kokkinos I, Murphy K, Yuille A L. Deeplab: Semantic image segmentation with deep convolutional nets, atrous convolution, and fully connected crfs. IEEE Trans Pattern Anal Mach Intell. 2017; 40(4):834-848.
25. Fitzpatrick J M, West J B. The distribution of target registration error in rigid-body point-based registration. IEEE Trans Med Imaging. 2001; 20(9):917-927.
26. Bland J M, Altman D. Statistical methods for assessing agreement between two methods of clinical measurement. The lancet. 1986; 327(8476):307-310.
27. Bland M. Is there a method I can use to assess the differences in limits of agreement between groups? Accessed Nov. 17, 2022.—users.york.ac.uk/~mb55/meas/comp_loa.htm
28. Dice L R. Measures of the amount of ecologic association between species. Ecology. 1945; 26(3):297-302.
29. Goodfellow I, Bengio Y, Courville A. Deep Learning. MIT press; 2016.
30. Sebek J, Albin N, Bortel R, Natarajan B, Prakash P. Sensitivity of microwave ablation models to tissue biophysical properties: A first step toward probabilistic modeling and treatment planning. Med Phys. 2016; 43(5):2649-2661.
31. Ahmed M, Solbiati L, Brace C L, et al. Image-guided tumor ablation: standardization of terminology and reporting criteria—a 10-year update. Journal of Vascular and Interventional Radiology. 2014; 25 (11): 1691-1705.
32. Moussa A M, Ziv E, Solomon S B, Camacho J C. Microwave ablation in primary lung malignancies. In: Seminars in Interventional Radiology. Vol 36. Thieme Medical Publishers; 2019:326-333.
33. Yan P, Tong A na, Nie X li, Ma M ge. Assessment of safety margin after microwave ablation of stage I NSCLC with three-dimensional reconstruction technique using C T imaging. BMC Med Imaging. 2021; 21(1):1-11.
34. Vasiniotis Kamarinos N, Vakiani E, Gonen M, et al. Biopsy and Margins Optimize Outcomes after Thermal Ablation of Colorectal Liver Metastases. Cancers (Basel). 2022; 14(3):693.
35. Sotirchos V S, Petrovic L M, Gönen M, et al. Colorectal cancer liver metastases: biopsy of the ablation zone and margins can be used to predict oncologic outcome. Radiology. 2016; 280(3):949.

1. A method of training models to determine ablation regions from biomedical images of subjects, comprising:
identifying, by a computing system, for a subject under treatment for lung cancer:
(i) a first biomedical image having a first region of interest (ROI) corresponding to a tumor in a lung region prior to an ablation procedure,
(ii) a second biomedical image having a second ROI corresponding to an administration of the ablation procedure in the lung region,
(iii) a parameter defining the administration of the ablation procedure to the tumor in the lung region, and
(iv) an annotation defining a first ablation region identifying a segment within the second biomedical image corresponding to the administration of the ablation procedure;
registering, by the computing system, the first biomedical image with the second biomedical image using the first ROI and the second ROI to generate a third biomedical image;
applying, by the computing system, a machine learning (ML) to the third biomedical image and the parameter to determine a second ablation region to identify a segment within the second biomedical image corresponding to the administration of the ablation procedure;
determining, by the computing system, a loss metric based on a comparison between the first ablation region and the second ablation region; and
updating, by the computing system, at least one weight of the ML model in accordance with the loss metric.

2. The method of claim 1, further comprising converting, by the computing system, prior to applying the ML model, the third biomedical image and the parameter to a coordinate system defined relative to an applicator within the lung region to administer the ablation procedure.

3. The method of claim 1, further comprising generating, by the computing system, within the third biomedical image, an application region identifying an application of the ablation procedure in the lung region based on the parameter, and wherein the applying the ML model further comprises applying the ML model to the application region to determine the second ablation region.

4. The method of claim 1, wherein registering further comprises constraining the registration of the first biomedical image with the second biomedical image as a function of a first anatomical structure corresponding to the first ROI, a second anatomical structure corresponding to the second ROI, and the administration of the ablation procedure.

5. The method of claim 1, wherein registering further comprises registering the first biomedical image with the second biomedical image in accordance with a sequence of image registration operations, the sequence of image registration operations including one or more of:
an affine registration between the first biomedical image and the second biomedical image,
a first deformable image registration between the first biomedical image and the second biomedical image,
a second deformable image registration between a first portion of the first biomedical image corresponding to the lung region and a second portion of the second biomedical image corresponding to the lung region,
a third deformable image registration between the first ROI of the first biomedical image and the second ROI of the second biomedical image, or
a landmark-based registration between a first portion of the first biomedical image corresponding to a first anatomical structure in the lung region and a second portion of the second biomedical image corresponding to a second anatomical structure in the lung region.

6. The method of claim 1, wherein the annotation identifies the first ablation region does not correspond to any anatomical boundary associated with the lung region of the subject.

7. (canceled)

8. The method of claim 1, wherein the parameter comprises at least one of: (i) a position of an applicator within the first biomedical image, (ii) a power to apply to the tumor, (iii) a duration of the administration of the ablation procedure, or (iv) a trajectory of an applicator through the lung region, and
wherein the ablation procedure comprises at least one of: (i) a radiofrequency ablation (RFA), (ii) a microwave ablation (MWA), or (iii) a cryoablation.

9. A system of training models to determine ablation regions from biomedical images of subjects, comprising:
a computing system having one or more processors coupled with memory, configured to:
identify for a subject under treatment for lung cancer:
a first biomedical image having a first region of interest (ROI) corresponding to a tumor in a lung region prior to an ablation procedure,
a second biomedical image having a second ROI corresponding to an administration of the ablation procedure in the lung region,
a parameter defining the administration of the ablation procedure to the tumor in the lung region, and
an annotation defining a first ablation region identifying a segment within the second biomedical image corresponding to the administration of the ablation procedure;
register the first biomedical image with the second biomedical image using the first ROI and the second ROI to generate a third biomedical image;
apply a machine learning (ML) to the third biomedical image and the parameter to determine a second ablation region to identify a segment within the second biomedical image corresponding to the administration of the ablation procedure;
determine a loss metric based on a comparison between the first ablation region and the second ablation region; and
update at least one weight of the ML model in accordance with the loss metric.

10. The system of claim 9, wherein the computing system is further configured to convert, prior to applying the ML model, the third biomedical image and the parameter to a coordinate system defined relative to an applicator within the lung region to administer the ablation procedure.

11. The system of claim 9, wherein the computing system is further configured to:
generate, within the third biomedical image, an application region identifying an application of the ablation procedure in the lung region based on the parameter, and
apply the ML model to the application region to determine the second ablation region.

12. The system of claim 9, wherein the computing system is further configured to constrain the registration of the first biomedical image with the second biomedical image as a function of a first anatomical structure corresponding to the first ROI, a second anatomical structure corresponding to the second ROI, and the administration of the ablation procedure.

13. The system of claim 9, wherein the computing system is further configured to register the first biomedical image with the second biomedical image in accordance with a sequence of image registration operations, the sequence of image registration operations including one or more of:
an affine registration between the first biomedical image and the second biomedical image,
a first deformable image registration between the first biomedical image and the second biomedical image,
a second deformable image registration between a first portion of the first biomedical image corresponding to the lung region and a second portion of the second biomedical image corresponding to the lung region,
a third deformable image registration between the first ROI of the first biomedical image and the second ROI of the second biomedical image, or
a landmark-based registration between a first portion of the first biomedical image corresponding to a first anatomical structure in the lung region and a second portion of the second biomedical image corresponding to a second anatomical structure in the lung region.

14. The system of claim 9, wherein the annotation identifies the first ablation region does not correspond to any anatomical boundary associated with the lung region of the subject.

15.-24. (canceled)

25. A method of determining ablation regions from biomedical images of subjects, comprising:
identifying, by a computing system, for a first subject under treatment for lung cancer:
a first biomedical image having a first region of interest (ROI) corresponding to a tumor in a lung region prior to an ablation procedure, and
a parameter defining an administration of the ablation procedure to the tumor within the lung region, and
applying, by the computing system, a machine learning (ML) model to the first biomedical image and the parameter to determine an ablation region to identify a segment within the first biomedical image corresponding to an administration of the ablation procedure;
storing, by the computing system, using one or more data structures, an association between the first subject and the ablation region.

26. The method of claim 25, further comprising converting, by the computing system, prior to applying the ML model, the first biomedical image and the parameter to a coordinate system defined relative to an applicator within the lung region to administer the ablation procedure.

27. The method of claim 25, further comprising generating, by the computing system, within the first biomedical image, an application region identifying an application of the ablation procedure in the lung region based on the parameter, and wherein the applying the ML model further comprises applying the ML model to the application region to determine the ablation region.

28. The method of claim 25, further comprising providing, by the computing system, information for presentation based on the association between the first subject and the ablation region.

29. The method of claim 25, wherein identifying further comprises selecting, via an interface, the parameter from a plurality of parameters to determine the ablation region in the subject in need of the ablation procedure, prior to the administration of the ablation procedure.

30. The method of claim 25, wherein the parameter comprises at least one of: (i) a position of an applicator within the first biomedical image, (ii) a power to apply to the tumor during the ablation procedure, (iii) a duration of the administration of the ablation procedure, or (iv) a trajectory of the applicator through the lung region, and
wherein the ablation procedure comprises at least one of: (i) a radiofrequency ablation (RFA), (ii) a microwave ablation (MWA), or (iii) a cryoablation.

31. The method of claim 25, further comprising performing the ablation procedure in accordance with the determined ablation region.

32.-43. (canceled)
